SEQUENCE LISTING (24 sequences)

SEQ ID NO: 1 - DNA, 111 bp, Homo sapiens
ttgggggccccttccccacactatctcaatgcaaatatctgtctgaaacggtccctggctaaactccacccatgggttggccagccttgccttgaccaatagccttgacaa

SEQ ID NO: 2 - RNA, 20 nt, Artificial
  Synthetic guide RNA
auugagauaguguggggaag

SEQ ID NO: 3 - RNA, 20 nt, Artificial
  Synthetic guide RNA
cauugagauaguguggggaa

SEQ ID NO: 4 - RNA, 20 nt, Artificial
  Synthetic guide RNA
gcauugagauagugugggga

SEQ ID NO: 5 - DNA, 30 bp, Artificial
  Synthetic primer
aaaaacggctgacaaaagaagtcctggtat

SEQ ID NO: 6 - DNA, 30 bp, Artificial
  Synthetic primer
ataacctcagacgttccagaagcgagtgtg

SEQ ID NO: 7 - DNA, 20 bp, Artificial
  Synthetic primer
tactgcgctgaaactgtggc

SEQ ID NO: 8 - DNA, 24 bp, Artificial
  Synthetic primer
ggcgtctggactaggagcttattg

SEQ ID NO: 9 - DNA, 26 bp, Artificial
  Synthetic primer
gcactgaaactgttgctttataggat

SEQ ID NO: 10 - DNA, 24 bp, Artificial
  Synthetic primer
ggcgtctggactaggagcttattg

SEQ ID NO: 11 - DNA, 20 bp, Artificial
  Synthetic primer
cagcaccaggatcagtgaaa

SEQ ID NO: 12 - DNA, 22 bp, Artificial
  Synthetic primer
ctatgtccacttcaggacagca

SEQ ID NO: 13 - DNA, 21 bp, Artificial
  Synthetic primer
cggtcaacttcaagctcctaa

SEQ ID NO: 14 - DNA, 20 bp, Artificial
  Synthetic primer
acagaagccaggaacttgtc

SEQ ID NO: 15 - DNA, 22 bp, Artificial
  Synthetic primer
gcaaggtgaacgtggatgaagt

SEQ ID NO: 16 - DNA, 23 bp, Artificial
  Synthetic primer
taacagcatcaggagtggacaga

SEQ ID NO: 17 - DNA, 19 bp, Artificial
  Synthetic primer
cctgtcctctgcctctgcc

SEQ ID NO: 18 - DNA, 19 bp, Artificial
  Synthetic primer
ggattgccaaaacggtcac

SEQ ID NO: 19 - DNA, 19 bp, Artificial
  Synthetic primer
caagggcactttttctcag

SEQ ID NO: 20 - DNA, 19 bp, Artificial
  Synthetic primer
aattccttgccaaagttgc

SEQ ID NO: 21 - DNA, 20 bp, Artificial
  Synthetic primer
tgctcctgggagtagattgg

SEQ ID NO: 22 - DNA, 20 bp, Artificial
  Synthetic primer
tggtatggggccaagagata

SEQ ID NO: 23 - DNA, 21 bp, Artificial
  Synthetic primer
acaagcctgtggggcaaggtg

SEQ ID NO: 24 - DNA, 20 bp, Artificial
  Synthetic primer
gccaggcacagggtccttcc

(19) United States
(12) Patent Application Publication
(10) Pub. No.: US 2022/0033856 A1
(43) Pub. Date: Feb. 3, 2022

(54) METHODS FOR INCREASING FETAL HEMOGLOBIN CONTENT IN EUKARYOTIC CELLS AND USES THEREOF FOR THE TREATMENT OF HEMOGLOBINOPATHIES

(71) Applicants: INSERM (Institut National de la Santé et de la Recherche Médicale), Paris (FR); Université de Paris, Paris (FR); Assistance Publique-Hôpitaux de Paris (APHP), Paris (FR); Fondation Imagine, Paris (FR)

(72) Inventors: Annarita MICCIO, Paris (FR); Leslie WEBER, Paris (FR)

(21) Appl. No.: 17/274,966

(22) PCT Filed: Sep. 10, 2019
(86) PCT No.: PCT/EP2019/074131

§ 371 Date: Mar. 10, 2021

(30) Foreign Application Priority Data
Sep. 11, 2018 (EP) 18306191.0

(51) Int. Cl. C12N 15/90; A61K 35/28; C12N 15/11; C12N 9/22

(52) U.S. Cl. CPC C12N 15/907; A61K 35/28; C12N 2800/80; C12N 9/22; C12N 2310/20; C12N 15/11

(57) ABSTRACT

The clinical severity of β-hemoglobinopathies is alleviated by the co-inheritance of genetic mutations causing a sustained fetal γ-globin chain production at adult age, a condition termed hereditary persistence of fetal hemoglobin (HPFH). Here, the inventors have compared the extent of fetal hemoglobin (HbF) de-repression following CRISPR/Cas9-mediated targeting of different regions of the HBG1 and HBG2 promoters in an adult erythroid cell line (HUDEP-2). They achieved a potent and pancellular HbF re-activation upon disruption of binding sites for γ-globin repressors located in both HBG1 and HBG2 genes. They validated these findings in Red Blood Cells (RBCs) derived from genome edited Sickle Cell Disease (SCD) patient hematopoietic stem/progenitor cells. Overall, this study identified a binding site for an HbF repressor as a novel and potent target for the treatment of β-hemoglobinopathies. Accordingly, the present invention relates to a method for increasing fetal hemoglobin content in a eukaryotic cell comprising the step of disrupting the binding site for Leukemia/lymphoma-related factor (LRF) in the HBG1 or HBG2 promoter.

### FIELD OF THE INVENTION

The present invention relates to methods for increasing fetal hemoglobin content in eukaryotic cells and uses thereof for the treatment of hemoglobinopathies.

### BACKGROUND OF THE INVENTION

β-hemoglobinopathies (β-thalassemia and Sickle Cell Disease (SCD)), the most prevalent genetic disorders worldwide, are caused by mutations affecting quantitatively or qualitatively the production of the adult hemoglobin (Hb) β-globin chain encoded by the HBB gene. In β-thalassemia, the reduced production of β-chains causes α-globin precipitation, ineffective erythropoiesis and insufficiently hemoglobinized red blood cells (RBCs). In SCD, the β6^{Glu→Val} substitution leads to Hb polymerization and RBC sickling, which is responsible for vaso-occlusive crises, hemolytic anemia and organ damage.

The clinical severity of β-hemoglobinopathies is alleviated by the co-inheritance of genetic mutations causing a sustained fetal γ-globin chain production at adult age, a condition termed hereditary persistence of fetal hemoglobin (HPFH; ref¹). Elevated fetal γ-globin levels reduces globin chain imbalance in β-thalassemias and exert a potent anti-sickling effect in SCD.

HPFH is caused by two different types of mutations: (i) large genomic deletions including the β- and δ-genes; (ii) mutations in the γ-globin promoters¹. The promoters of the two human γ-globin genes HBG1 and HBG2 are identical up to position −221 nt from the transcription start site (TSS). HPFH-associated mutations in the γ-globin gene promoters are clustered in three regions located ˜115, 175 and 200 nt upstream of the HBG TSS² (FIG. 1A). These mutations are associated to high levels of HbF (accounting up to 40% of total Hb) in adult life and are thought either to generate de novo DNA motifs recognized by transcriptional activators or to disrupt binding sites for transcriptional repressors.

For instance, the −198 T>C point mutation in the HBG1 promoter, also called British-type HPFH (5 to 20% HbF levels), has been recently shown to create a de novo binding site for the potent erythroid transcriptional activator Krüppel-like factor 1 (KLF1)³. Similarly, an HPFH point mutation (T>C) at position −175 nt of both the γ-globin promoters (17 to 38% HbF levels) creates a binding site for TAL1, a transcription factor activating the expression of many erythroid-specific genes⁴.

The −115 and −200 regions contain the greatest variety of described HPFH mutations (FIG. 1A), and have been recently shown to recruit the transcriptional repressors LRF and BCL11A, respectively^{2,5}. BCL11A and LRF exert their repressive activity through the Nucleosome Remodeling and Deacetylase repressor complex (NuRD complex)⁶, which contains numerous proteins and notably histone deacetylases (HDAC 1 and 2). These proteins carry enzymatic activities that induce post-translational histone deacetylation, thus maintaining a closed chromatin conformation. Importantly, LRF and BCL11A occupancy at the γ-globin promoters decreased in HUDEP-2 cells harboring the −195 C>G and −114 C>T HPFH mutations, respectively. Similarly, a CRISPR/Cas9-based approach leading to the generation of a naturally occurring HPFH 13-nt deletion spanning the BCL11A binding site caused a significant increase of γ-globin expression in both HUDEP-2 cells and primary hematopoietic stem progenitor cell (HSPC)-derived RBCs⁷.

Besides HPFH mutations, Single Nucleotide Polymorphisms (SNPs) have been described at the −158 nt position of both γ-globin promoters; these variants are associated with enhanced γ-globin expression under conditions of stress erythropoiesis, e.g., in SCD and β-thalassemia (FIG. 1A, ref⁸⁻¹⁰).

Recreating the naturally occurring HPFH point mutations described in patients by using the high-fidelity Homologous Direct Repair (HDR) mechanism would represent an ideal strategy to ameliorate the phenotype of SCD and β-thalassemic patients by inducing fetal hemoglobin expression. However, HDR is known to be inefficient in Hematopoietic Stem Cells (HPSCs), Non Homologous End Joining (NHEJ) being the most prevalent repair mechanism in quiescent cells¹¹.

### SUMMARY OF THE INVENTION

The present invention relates to methods for increasing fetal hemoglobin content in eukaryotic cells and uses thereof for the treatment of hemoglobinopathies. In particular, the present invention is defined by the claims.

### DETAILED DESCRIPTION OF THE INVENTION

The clinical severity of β-hemoglobinopathies is alleviated by the co-inheritance of genetic mutations causing a sustained fetal γ-globin chain production at adult age, a condition termed hereditary persistence of fetal hemoglobin (HPFH). Naturally occurring HPFH point mutations identified in the promoters of the two γ-globin genes, HBG1 and HBG2, cluster at several loci, and are thought either to generate de novo DNA motifs recognized by transcription activators or disrupt binding sites for transcriptional repressors. Here, the inventors have compared the extent of fetal hemoglobin (HbF) de-repression following CRISPR/Cas9-mediated targeting of different regions of the HBG1 and HBG2 promoters in an adult erythroid cell line (HUDEP-2). They achieved a potent and pancellular HbF re-activation upon disruption of binding sites for γ-globin repressors located in both HBG1 and HBG2 genes. They validated these findings in Red Blood Cells (RBCs) derived from genome edited Sickle Cell Disease (SCD) patient hematopoietic stem/progenitor cells. Overall, this study identified a binding site for an HbF repressor as a novel and potent target for the treatment of β-hemoglobinopathies.

Accordingly, the first object of the present invention relates to a method for increasing fetal hemoglobin content in a eukaryotic cell comprising the step of disrupting the binding site for Leukemia/lymphoma-related factor (LRF) in the HBG1 or HBG2 promoter.

In some embodiments, the method of the present invention comprises contacting the eukaryotic cell with an effective amount of a DNA-targeting endonuclease whereby the DNA-targeting endonuclease cleaves the genomic DNA of the cell in at least one position located in or close to the binding site for Leukemia/lymphoma-related factor (LRF) in the HBG1 or HBG2 promoters.

In some embodiments, the eukaryotic cell is selected from the group consisting of hematopoietic progenitor cells, hematopoietic stem cells (HSCs), pluripotent cells (i.e. embryonic stem cells (ES) and induced pluripotent stem cells (iPS)).

Typically, the eukaryotic cell results from a stem cell mobilization. As used herein, the term “mobilization” or “stem cell mobilization” refers to a process involving the recruitment of stem cells from their tissue or organ of residence to peripheral blood following treatment with a mobilization agent. This process mimics the enhancement of the physiological release of stem cells from tissues or organs in response to stress signals during injury and inflammation. The mechanism of the mobilization process depends on the type of mobilization agent administered. Some mobilization agents act as agonists or antagonists that prevent the attachment of stem cells to cells or tissues of their microenvironment. Other mobilization agents induce the release of proteases that cleave the adhesion molecules or support structures between stem cells and their sites of attachment. As used herein, the term “mobilization agent” refers to a wide range of molecules that act to enhance the mobilization of stem cells from their tissue or organ of residence, e.g., bone marrow (e.g., CD34+ stem cells) and spleen (e.g., Hox11+ stem cells), into peripheral blood. Mobilization agents include chemotherapeutic drugs, e.g., cyclophosphamide and cisplatin; cytokines, and chemokines, e.g., granulocyte colony-stimulating factor (G-CSF), granulocyte-macrophage colony-stimulating factor (GM-CSF), stem cell factor (SCF), Fms-related tyrosine kinase 3 (flt-3) ligand, stromal cell-derived factor 1 (SDF-1); agonists of the chemokine (C-C motif) receptor 1 (CCR1), such as chemokine (C-C motif) ligand 3 (CCL3, also known as macrophage inflammatory protein-1α (Mip-1α)); agonists of the chemokine (C-X-C motif) receptor 1 (CXCR1) and 2 (CXCR2), such as chemokine (C-X-C motif) ligand 2 (CXCL2) (also known as growth-related oncogene protein-β (Gro-β)), and CXCL8 (also known as interleukin-8 (IL-8)); agonists of CXCR4, such as CTCE-02142, and Met-SDF-1; Very Late Antigen (VLA)-4 inhibitors; antagonists of CXCR4, such as TG-0054, plerixafor (also known as AMD3100), and AMD3465, or any combination of the previous agents. A mobilization agent increases the number of stem cells in peripheral blood, thus allowing for a more accessible source of stem cells for use in transplantation, organ repair or regeneration, or treatment of disease.

As used herein, the term “hematopoietic stem cell” or “HSC” refers to blood cells that have the capacity to self-renew and to differentiate into precursors of blood cells. These precursor cells are immature blood cells that cannot self-renew and must differentiate into mature blood cells. Hematopoietic stem progenitor cells display a number of phenotypes, such as Lin−CD34+CD38−CD90+CD45RA−, Lin−CD34+CD38−CD90−CD45RA−, Lin−CD34+CD38+IL-3aloCD45RA−, and Lin−CD34+CD38+CD10+ (Daley et al., Focus 18:62-67, 1996; Pimentel, E., Ed., Handbook of Growth Factors Vol. III: Hematopoietic Growth Factors and Cytokines, pp. 1-2, CRC Press, Boca Raton, Fla., 1994). Within the bone marrow microenvironment, the stem cells self-renew and maintain continuous production of hematopoietic stem cells that give rise to all mature blood cells throughout life. In some embodiments, the hematopoietic progenitor cells or hematopoietic stem cells are isolated form peripheral blood cells.

As used herein, the term “peripheral blood cells” refer to the cellular components of blood, including red blood cells, white blood cells, and platelets, which are found within the circulating pool of blood. In some embodiments, the eukaryotic cell is a bone marrow derived stem cell.

As used herein the term “bone marrow-derived stem cells” refers to stem cells found in the bone marrow. Stem cells may reside in the bone marrow, either as an adherent stromal cell type that possess pluripotent capabilities, or as cells that express CD34 or CD45 cell-surface protein, which identifies hematopoietic stem cells able to differentiate into blood cells.

Typically, the eukaryotic cell is isolated. As used herein, the term “isolated cell” refers to a cell that has been removed from an organism in which it was originally found, or a descendant of such a cell. Optionally the eukaryotic cell has been cultured in vitro, e.g., in the presence of other cells. Optionally the eukaryotic cell is later introduced into a second organism or reintroduced into the organism from which it (or the cell from which it is descended) was isolated. As used herein, the term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched.

As used herein the term “increasing the fetal hemoglobin content” in a cell indicates that fetal hemoglobin is at least 5% higher in the eukaryotic cell treated with the DNA-targeting endonuclease, than in a comparable, eukaryotic cell, wherein an endonuclease targeting an unrelated locus is present or where no endonuclease is present. In some embodiments, the percentage of fetal hemoglobin expression in the eukaryotic cell is at least 10% higher, at least 20% higher, at least 30% higher, at least 40% higher, at least 50% higher, at least 60% higher, at least 70% higher, at least 80% higher, at least 90% higher, at least 1-fold higher, at least 2-fold higher, at least 5-fold higher, at least 10 fold higher, at least 100 fold higher, at least 1000-fold higher, or more than an eukaryotic cell, wherein an endonuclease targeting an unrelated locus is present or where no endonuclease is present. In some embodiments, any method known in the art can be used to measure an increase in fetal hemoglobin expression, e. g. HPLC analysis of fetal γ-globin protein and RT-qPCR analysis of fetal γ-globin mRNA. Typically, said methods are described in the EXAMPLE.

As used herein, the term “gamma globin” or “γ-globin” has its general meaning in the art and refers to protein that is encoded in human by the HBG1 and HBG2 genes. The HBG1 and HBG2 genes are normally expressed in the fetal liver, spleen and bone marrow. Two γ-globin chains together with two α-globin chains constitute fetal hemoglobin (HbF) which is normally replaced by adult hemoglobin (HbA) in the year following birth (Higgs D R, Vickers M A, Wilkie A O, Pretorius I M, Jarman A P, Weatherall D J (May 1989). “A review of the molecular genetics of the human alpha-globin gene cluster”. Blood. 73 (5): 1081-104.). The ENSEMBL IDs (i.e. the gene identifier number from the Ensembl Genome Browser database) for HBG1 and HBG2 are ENSG00000213934 and ENSG00000196565 respectively.

As used herein, the term “promoter” has its general meaning in the art and refers to a nucleic acid sequence which is required for expression of a gene operably linked to the promoter sequence. HBG1 and HBG2 promoters are identical up to −221 bp and comprise the nucleic acid sequence as set forth in SEQ ID NO:1 and depicted in FIG. 1A. According to the present invention, the first nucleotide in SEQ ID NO:1 denotes the nucleotide located at position −210 upstream of the HBG transcription starting site and the last nucleotide in SEQ ID NO:1 denotes the nucleotide located at position −100 upstream of the HBG transcription starting site. Accordingly and inversely:
the nucleotide at position −197 in the HBG1 or HBG2 promoter denotes the nucleotide at position 14 in SEQ ID NO:1,
the nucleotide at position −196 in the HBG1 or HBG2 promoter denotes the nucleotide at position 15 in SEQ ID NO:1, and,
the nucleotide at position −195 in the HBG1 or HBG2 promoter denotes the nucleotide at position 16 in SEQ ID NO:1.
   According to the present invention the “−200 region” in the HBG1 or HBG2 promoter refers to the region which encompasses the nucleotides at position −197; −196 and −195 and thus relates to the region starting from the nucleotide at position 11 (i.e. −200) to the nucleotide at position 21 (i.e. −190) in SEQ ID NO:1, and more preferably to the region starting from the nucleotide at position 14 to the nucleotide at position 16 in SEQ ID NO:1.

SEQ ID NO 1:
TTGGGGGCCCCTTCCCCACACTATCTCAATGCAAATAT
CTGTCTGAAACGGTCCCTGGCTAAACTCCACCCATGGG
TTGGCCAGCCTTGCCTTGACCAATAGCCTTGACAA

As used herein, the term “LRF” has its general meaning in the art and refers to the transcriptional repressor, which is Leukemia/lymphoma-related factor (LRF), encoded by the ZBTB7A gene. LRF is a ZBTB transcription factor that binds DNA through C-terminal C2H2-type zinc fingers and presumably recruits a transcriptional repressor complex through its N-terminal BTB domain (Lee S U, Maeda T. Immunol. Rev. 2012; 247:107-119). Accordingly, the term “transcriptional repressor binding site” refers to a site present on DNA whereby the transcription repressor binds. In some embodiments, the DNA-targeting endonuclease of the present invention edits the genome sequence of the eukaryotic cell so that the transcriptional repressor is not able to bind to its transcriptional repressor binding sites. In some embodiments, the DNA-targeting endonuclease of the present invention will inhibit the binding of LRF to its binding sites.

As used herein, the term “DNA targeting endonuclease” has its general meaning in the art and refers to an endonuclease that generates a double-strand break (DSB) at a desired position in the genome without producing undesired toxic off-target DSBs. The DNA targeting endonuclease can be a naturally occurring endonuclease (e.g., a bacterial meganuclease) or it can be artificially generated (e.g., engineered meganucleases, TALENs, or ZFNs, among others).

As used herein the term “cleaves” generally refers to the generation of a double-strand break in the DNA genome at a desired location. The term “cleavage site” refers to any site in a target sequence that can be cleaved by a DNA targeting endonuclease. Cleavage thus results in alteration of the genome sequence by non-homologous end joining (NHEJ) repair system or microhomology mediated end joining (MMEJ) repair system. According to the present invention alteration by NHEJ repair system is preferred. The term “alteration” or “genome editing” of the genomic sequence includes a replacement of one or more nucleotides, the insertion of one or more nucleotides, and/or the deletion of one or more nucleotides anywhere within a genome.

In some embodiments, the DNA-targeting endonuclease leads to the genome editing of the −200 region in in the HBG1 or HBG2 promoter.

In some embodiments, the DNA targeting endonuclease of the present invention cleaves the genomic sequence between positions −198 and −197 in the HBG1 or HBG2 promoter (i.e. cleaves the genomic sequence between positions 13 and 14 in SEQ ID NO:1).

In some embodiments, the DNA targeting endonuclease of the present invention cleaves the genomic sequence between positions −197 and −196 in the HBG1 or HBG2 promoter (i.e. cleaves the genomic sequence between positions 14 and 15 in SEQ ID NO:1).

In some embodiments, the DNA targeting endonuclease of the present invention cleaves the genomic sequence between positions −196 and −195 in the HBG1 or HBG2 promoter (i.e. cleaves the genomic sequence between positions 15 and 16 in SEQ ID NO:1).

In some embodiments, the DNA targeting endonuclease of the present invention is a TALEN. As used herein, the term “TALEN” has its general meaning in the art and refers to a transcription activator-like effector nuclease, an artificial nuclease which can be used to edit a target gene. TALENs are produced artificially by fusing a TAL effector (“TALE”) DNA binding domain, e.g., one or more TALEs, e.g., 1, 2, 3, 4, 5, 6, 7, 8, 9 or 10 TALEs to a DNA-modifying domain, e.g., a FokI nuclease domain. Transcription activator-like effects (TALEs) can be engineered to bind any desired DNA sequence (Zhang (2011), Nature Biotech. 29: 149-153). By combining an engineered TALE with a DNA cleavage domain, a restriction enzyme can be produced which is specific to any desired DNA sequence. These can then be introduced into a cell, wherein they can be used for genome editing (Boch (2011) Nature Biotech. 29: 135-6; and Boch et al. (2009) Science 326: 1509-12; Moscou et al. (2009) Science 326: 3501). TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence (Zhang (2011), Nature Biotech. 29: 149-153). To produce a TALEN, a TALE protein is fused to a nuclease (N), e.g., a wild-type or mutated FokI endonuclease. Several mutations to FokI have been made for its use in TALENs; these, for example, improve cleavage specificity or activity (Cermak et al. (2011) Nucl. Acids Res. 39: e82; Miller et al. (2011) Nature Biotech. 29: 143-8; Hockemeyer et al. (2011) Nature Biotech. 29: 731-734; Wood et al. (2011) Science 333: 307; Doyon et al. (2010) Nature Methods 8: 74-79; Szczepek et al. (2007) Nature Biotech. 25: 786-793; and Guo et al. (2010) J. Mol. Biol. 200: 96). The Fold domain functions as a dimer, requiring two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity (Miller et al. (2011) Nature Biotech. 29: 143-8). TALEN can be used inside a cell to produce a double-strand break in a target nucleic acid, e.g., a site within a gene. A mutation can be introduced at the break site if the repair mechanisms improperly repair the break via non-homologous end joining (Huertas, P., Nat. Struct. Mol. Biol. (2010) 17: 11-16). For example, improper repair may introduce a frame shift mutation. Alternatively, foreign DNA can be introduced into the cell along with the TALEN; depending on the sequences of the foreign DNA and chromosomal sequence, this process can be used to modify a target gene via the homologous direct repair pathway, e.g., correct a defect in the target gene, thus causing expression of a repaired target gene, or e.g., introduce such a defect into a wt gene, thus decreasing expression of a target gene.

In some embodiments, the DNA targeting endonuclease of the present invention is a ZFN. As used herein, the term “ZFN” or “Zinc Finger Nuclease” has its general meaning in the art and refers to a zinc finger nuclease, an artificial nuclease which can be used to edit a target gene. Like a TALEN, a ZFN comprises a DNA-modifying domain, e.g., a nuclease domain, e.g., a Fold nuclease domain (or derivative thereof) fused to a DNA-binding domain. In the case of a ZFN, the DNA-binding domain comprises one or more zinc fingers, e.g., 1, 2, 3, 4, 5, 6, 7, 8, 9 or 10 zinc fingers (Carroll et al. (2011) Genetics Society of America 188: 773-782; and Kim et al. (1996) Proc. Natl. Acad. Sci. USA 93: 1156-1160). A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art (Sera (2002), Biochemistry, 41:7074-7081; Liu (2008) Bioinformatics, 24:1850-1857). A ZFN using a FokI nuclease domain or other dimeric nuclease domain functions as a dimer. Thus, a pair of ZFNs are required to target non-palindromic DNA sites. The two individual ZFNs must bind opposite strands of the DNA with their nucleases properly spaced apart (Bitinaite et al. (1998) Proc. Natl. Acad. Sci. USA 95: 10570-5). Also like a TALEN, a ZFN can create a DSB in the DNA, which can create a frame-shift mutation if improperly repaired, e.g., via non-homologous end joining, leading to a decrease in the expression of a target gene in a cell.

In some embodiments, the DNA targeting endonuclease of the present invention is a CRISPR-associated endonuclease. As used herein, the term “CRISPR-associated endonuclease” has its general meaning in the art and refers to clustered regularly interspaced short palindromic repeats associated which are the segments of prokaryotic DNA containing short repetitions of base sequences. In bacteria the CRISPR/Cas loci encode RNA-guided adaptive immune systems against mobile genetic elements (viruses, transposable elements and conjugative plasmids). Three types (I-VI) of CRISPR systems have been identified. CRISPR clusters contain spacers, the sequences complementary to antecedent mobile elements. CRISPR clusters are transcribed and processed into mature CRISPR (Clustered Regularly Interspaced Short Palindromic Repeats) RNA (crRNA). The CRISPR-associated endonucleases Cas9 and Cpf1 belong to the type II and type V CRISPR/Cas system and have strong endonuclease activity to cut target DNA. Cas9 is guided by a mature crRNA that contains about 20 nucleotides of unique target sequence (called spacer) and a trans-activated small RNA (tracrRNA) that serves as a guide for ribonuclease Ill-aided processing of pre-crRNA. The crRNA:tracrRNA duplex directs Cas9 to target DNA via complementary base pairing between the spacer on the crRNA and the complementary sequence (called protospacer) on the target DNA. Cas9 recognizes a trinucleotide (NGG) protospacer adjacent motif (PAM) to specify the cut site (the 3^{rd} or the 4^{th} nucleotide from PAM). The crRNA and tracrRNA can be expressed separately or engineered into an artificial fusion small guide RNA (sgRNA) via a synthetic stem loop to mimic the natural crRNA/tracrRNA duplex. Such sgRNA, like shRNA, can be synthesized or in vitro transcribed for direct RNA transfection or expressed from U6 or H1-promoted RNA expression vector.

In some embodiments, the CRISPR-associated endonuclease is a Cas9 nuclease. The Cas9 nuclease can have a nucleotide sequence identical to the wild type Streptococcus pyrogenes sequence. In some embodiments, the CRISPR-associated endonuclease can be a sequence from other species, for example other Streptococcus species, such as thermophilus; Pseudomonas aeruginosa, Escherichia coli, or other sequenced bacteria genomes and archaea, or other prokaryotic microorganisms. Alternatively, the wild type Streptococcus pyogenes Cas9 sequence can be modified. The nucleic acid sequence can be codon optimized for efficient expression in mammalian cells, i.e., “humanized.” A humanized Cas9 nuclease sequence can be for example, the Cas9 nuclease sequence encoded by any of the expression vectors listed in Genbank accession numbers KM099231.1 GL669193757; KM099232.1 GL669193761; or KM099233.1 GL669193765. Alternatively, the Cas9 nuclease sequence can be for example, the sequence contained within a commercially available vector such as pX330, pX260 or pMJ920 from Addgene (Cambridge, Mass.). In some embodiments, the Cas9 endonuclease can have an amino acid sequence that is a variant or a fragment of any of the Cas9 endonuclease sequences of Genbank accession numbers KM099231.1 GL669193757; KM099232.1; GL669193761; or KM099233.1 GL669193765 or Cas9 amino acid sequence of pX330, pX260 or pMJ920 (Addgene, Cambridge, Mass.).

In some embodiments, the CRISPR-associated endonuclease is a Cpf1 nuclease. As used herein, the term “Cpf1 protein” to a Cpf1 wild-type protein derived from Type V CRISPR-Cpf1 systems, modifications of Cpf1 proteins, variants of Cpf1 proteins, Cpf1 orthologs, and combinations thereof. The cpf1 gene encodes a protein, Cpf1, that has a RuvC-like nuclease domain that is homologous to the respective domain of Cas9, but lacks the HNH nuclease domain that is present in Cas9 proteins. Type V systems have been identified in several bacteria, including Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA 33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237 (MbCpf1), Smithella spp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1). Recently it has been demonstrated that Cpf1 also has RNase activity and it is responsible for pre-crRNA processing (Fonfara, I., et al., “The CRISPR-associated DNA-cleaving enzyme Cpf1 also processes precursor CRISPR RNA,” Nature 28; 532(7600):517-21 (2016)).

In some embodiments, nucleotide sequence encoding for the nuclease (e.g. Cas9 or Cpf1) can be modified to encode biologically active variants of said nuclease, and these variants can have or can include, for example, an amino acid sequence that differs from a wild type nuclease by virtue of containing one or more mutations (e.g., an addition, deletion, or substitution mutation or a combination of such mutations). One or more of the substitution mutations can be a substitution (e.g., a conservative amino acid substitution). For example, a biologically active variant of a nuclease polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type nuclease polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The nuclease sequence can be a mutated sequence. For example the Cas9 nuclease can be mutated in the conserved FiNH and RuvC domains, which are involved in strand specific cleavage. For example, an aspartate-to-alanine (D10A) mutation in the RuvC catalytic domain allows the Cas9 nickase mutant (Cas9n) to nick rather than cleave DNA to yield single-stranded breaks.

In some embodiments, the method of the present invention comprises the step of contacting the eukaryotic cell with an effective amount of a CRISPR-associated endonuclease and with one or more guide RNA.

As used herein, the term “guide RNA” or “gRNA” has its general meaning in the art and refers to an RNA which can be specific for a target DNA and can form a complex with the CRISPR-associated endonuclease. A guide RNA can comprise a spacer sequence that specifies a target site and guides an RNA/Cas complex to a specified target DNA for cleavage. Site-specific cleavage of a target DNA occurs at locations determined by both 1) base-pairing complementarity between a guide RNA and a target DNA (also called a protospacer) and 2) a short motif in a target DNA referred to as a protospacer adjacent motif (PAM). The sequence of the PAM can vary depending upon the specificity requirements of the CRISPR endonuclease used. In the CRISPR-Cas system derived from S. pyogenes, the target DNA typically immediately precedes a 5′-NGG proto-spacer adjacent motif (PAM). Thus, for the S. pyogenes Cas9, the PAM sequence can be AGG, TGG, CGG or GGG. Other Cas9 orthologs may have different PAM specificities. The specific sequence of the guide RNA may vary, but, regardless of the sequence, useful guide RNA sequences will be those that minimize off-target effects while achieving high efficiency of alteration at the targeted loci. The length of the spacer sequence can vary from about 17 to about 60 or more nucleotides, for example about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 45, about 50, about 55, about 60 or more nucleotides. The guide RNA sequence can be configured as a single sequence or as a combination of one or more different sequences, e.g., a multiplex configuration. Multiplex configurations can include combinations of two, three, four, five, six, seven, eight, nine, ten, or more different guide RNAs.

In some embodiments, the guide RNA is used for recruiting the CRISPR-associated endonuclease to the HBG1 and HBG2 promoters and generating DSBs between positions −198 and −197 (i.e. between positions 13 and 14 in SEQ ID NO:1). In some embodiments, the guide RNA comprises a spacer sequence capable of annealing to the sequence ranging from the nucleotide at position −200 to the nucleotide at position −181 (i.e. ranging from the nucleotide at position 11 to the nucleotide at position 30 in SEQ ID NO:1). In some embodiments, the guide RNA comprises the spacer sequence as set forth in SEQ ID NO: 2 (5′ AUUGAGAUAGUGUGGGGAAG 3′) for recruiting the CRISPR-associated endonuclease to the HBG1 and HBG2 promoters and generating double-strand breaks between positions −198 and −197 (i.e. between positions 13 and 14 in SEQ ID NO:1).

In some embodiments, the guide RNA is used for recruiting the CRISPR-associated endonuclease to the HBG1 and HBG2 promoters and generating DSBs between positions −197 and −196 (i.e. between positions 14 and 15 in SEQ ID NO:1). In some embodiments, the guide RNA comprises a spacer sequence capable of annealing to the sequence ranging from the nucleotide at position −199 to the nucleotide at position −180 (i.e. ranging from the nucleotide at position 12 to the nucleotide at position 31 in SEQ ID NO:1). In some embodiments, the guide RNA comprises the spacer sequence as set forth in SEQ ID NO: 3 (5′ CAUUGAGAUAGUGUGGGGAA 3′) for recruiting the CRISPR-associated endonuclease to the HBG1 and HBG2 promoters and generating double-strand breaks between positions −197 and −196 (i.e. between positions 14 and 15 in SEQ ID NO:1).

In some embodiments, the guide RNA is used for recruiting the CRISPR-associated endonuclease to the HBG1 and HBG2 promoters and generating double-strand breaks between positions −195 and −196 (i.e. between positions 15 and 16 in SEQ ID NO:1). In some embodiments, the guide RNA comprises a spacer sequence capable of annealing to the sequence ranging from the nucleotide at position −198 to the nucleotide at position −179 (i.e. ranging from the nucleotide at position 13 to the nucleotide at position 32 in SEQ ID NO:1). In some embodiments, the guide RNA comprises the spacer sequence as set forth in SEQ ID NO: 4 (5′ GCAUUGAGAUAGUGUGGGGA 3′) for recruiting the CRISPR-associated endonuclease to the HBG1 and HBG2 promoters and generating double-strand breaks between positions −195 and −196 (i.e. between positions 15 and 16 in SEQ ID NO:1).

In some embodiments, the RNA molecule can be transcribed in vitro and/or can be chemically synthesized. The one skilled in the art can easily provide some modifications that will improve the clinical efficacy of the guide RNAs. Typically, chemical modifications include backbone modifications, heterocycle modifications, sugar modifications, and conjugations strategies. For example, the guide RNA may be stabilized. A “stabilized” RNA refers to RNA that is relatively resistant to in vivo degradation (e.g. via an exo- or endo-nuclease). Stabilization can be a function of length or secondary structure. In particular, RNA stabilization can be accomplished via phosphate backbone modifications. Chemical modifications that may be used in the practice of the invention include the following: Phosphorothioate groups, 5′ blocking groups (e.g., 5′ diguanosine caps), 3′ blocking groups, 2′-fluoro nucleosides, 2′-O-methyl-3′ phosphorothioate, or 2′-O-methyl-3′ thioPACE, inverted dT, inverted ddT, and biotin.

In some embodiments, the CRISPR-associated endonuclease and the guide RNA are provided to the population of eukaryotic cells through expression from one or more expression vectors. In some embodiments, the CRISPR endonuclease can be encoded by the same nucleic acid as the guide RNA sequences. Alternatively or in addition, the CRISPR endonuclease can be encoded in a physically separate nucleic acid from the guide RNA sequences or in a separate vector. Suitable vector backbones include, for example, those routinely used in the art such as plasmids, viruses, artificial chromosomes, BACs, YACs, or PACs. Suitable vectors include derivatives of SV40 and known bacterial plasmids, e.g., E. coli plasmids col E1, pCR1, pBR322, pMal-C2, pET, pGEX, pMB9 and their derivatives, plasmids such as RP4; phage DNAs, e.g., the numerous derivatives of phage 1, e.g., NM989, and other phage DNA, e.g., Ml 3 and filamentous single stranded phage DNA. Vectors also include, for example, viral vectors (such as adenoviruses (“Ad”), adeno-associated viruses (AAV), and vesicular stomatitis virus (VSV) and retroviruses), liposomes and other lipid-containing complexes, and other macromolecular complexes capable of mediating delivery of a polynucleotide to a host cell.

In some embodiments, the CRISPR-associated endonuclease can be pre-complexed with a guide RNA to form a ribonucleoprotein (RNP) complex. As used herein, the term “ribonucleoprotein complex,” or “ribonucleoprotein particle” refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid it is referred to as “ribonucleoprotein.” The interaction between the ribonucleoprotein and the ribonucleic acid may be direct, e.g., by covalent bond, or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). The RNP complex can thus be introduced into the eukaryotic cell. Introduction of the RNP complex can be timed. The cell can be synchronized with other cells at G1, S, and/or M phases of the cell cycle. RNP delivery avoids many of the pitfalls associated with mRNA, DNA, or viral delivery. Typically, the RNP complex is produced simply by mixing Cas9 and one or more guide RNAs in an appropriate buffer. This mixture is incubated for 5-10 min at room temperature before electroporation. Electroporation is a delivery technique in which an electrical field is applied to one or more cells in order to increase the permeability of the cell membrane. In some embodiments, genome editing efficiency can be improved by adding a transfection enhancer oligonucleotide.

A further object of the present invention relates to a method for increasing fetal hemoglobin levels in a subject in need thereof, the method comprising transplanting a therapeutically effective amount of a population of eukaryotic cells obtained by the method as above described.

In some embodiments, the population of cell is autologous to the subject, meaning the population of cells is derived from the same subject.

In some embodiments, the subject has been diagnosed with a hemoglobinopathy. The method of the present invention is thus particularly suitable for the treatment of hemoglobinopathies.

As used herein, the term “hemoglobinopathy” has its general meaning in the art and refers to any defect in the structure or function of any hemoglobin of an individual, and includes defects in the primary, secondary, tertiary or quaternary structure of hemoglobin caused by any mutation, such as deletion mutations or substitution mutations in the coding regions of the HBBgene, or mutations in, or deletions of, the promoters or enhancers of such gene that cause a reduction in the amount of hemoglobin produced as compared to a normal or standard condition. In some embodiments, the hemoglobinopathy is a β-hemoglobinopathy. In some embodiments, the β-hemoglobinopathy is a sickle cell disease. As used herein, “sickle cell disease” has its general meaning in the art and refers to a group of autosomal recessive genetic blood disorders, which results from mutations in a globin gene and which is characterized by red blood cells that assume an abnormal, rigid, sickle shape. They are defined by the presence of βS-globin gene coding for a β-globin chain variant in which glutamic acid is substituted by valine at amino acid position 6 of the peptide: incorporation of the βS-globin in the Hb tetramers (HbS, sickle Hb) leads to Hb polymerization and to a clinical phenotype. The term includes sickle cell anemia (HbSS), sickle-hemoglobin C disease (HbSC), sickle beta-plus-thalassemia (HbS/β+), or sickle beta-zero thalassemia (HbS/β0). In some embodiments, the hemoglobinopathy is a β-thalassemia. As used herein, the term “β-thalassemia” refers to a hemoglobinopathy that results from an altered ratio of α-globin to β-like globin polypeptide chains resulting in the underproduction of normal hemoglobin tetrameric proteins and the precipitation of free, unpaired α-globin chains.

By a “therapeutically effective amount” is meant a sufficient amount of population of cells to treat the disease at a reasonable benefit/risk ratio applicable to any medical treatment. It will be understood that the total usage compositions of the present invention will be decided by the attending physician within the scope of sound medical judgment. The specific therapeutically effective dose level for any particular patient will depend upon a variety of factors including the age, body weight, general health, sex and diet of the patient, the time of administration, route of administration, the duration of the treatment, drugs used in combination or coincidental with the population of cells, and like factors well known in the medical arts. In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the cells with the desired specificity, on the age and weight of the recipient, and on the severity of the targeted condition. These amount of cells can be as low as approximately 10³/kg, preferably 5×10³/kg; and as high as 10⁷/kg, preferably 10⁸/kg. The number of cells will depend upon the ultimate use for which the composition is intended, as will the type of cells included therein. Typically, the minimal dose is 2 million of cells per kg. Usually 2 to 20 million of cells are injected in the subject. The desired purity can be achieved by introducing a sorting step. For uses provided herein, the cells are generally in a volume of a liter or less, can be 500 ml or less, even 250 ml or 100 ml or less. The clinically relevant number of cells can be apportioned into multiple infusions that cumulatively equal or exceed the desired total amount of cells.

A further object of the present invention relates to a kit of parts comprising i) a CRISPR-associated endonuclease and ii) the guide RNA that comprises the sequence as set forth in SEQ ID NO:2.

A further object of the present invention relates to a kit of parts comprising i) a CRISPR-associated endonuclease and ii) the guide RNA that comprises the sequence as set forth in SEQ ID NO:3.

A further object of the present invention relates to a kit of parts comprising i) a CRISPR-associated endonuclease and ii) the guide RNA that comprises the sequence as set forth in SEQ ID NO:4.

A further object of the present invention relates to kit of parts of the present invention for use in a method for increasing fetal hemoglobin content in a eukaryotic cell.

A further object of the present invention relates to kit of parts of the present invention for use in a method for the treatment of a hemoglobinopathy in a subject in need thereof.

The invention will be further illustrated by the following figures and examples. However, these examples and figures should not be interpreted in any way as limiting the scope of the present invention.

### FIGURES

FIG. 1. HBG promoter disruption in HUDEP-2 cells leads to increased HbF levels. (A) Schematic representation of the β-globin locus on chromosome 11, depicting the Hypersensitive sites of the Locus Control Region (white boxes) and HBE1, HBG2, HBG1, HBD and HBB genes. The HBG2 and HBG1 promoter identical sequence from −210 to −100 nucleotides upstream the HBG TSSs is shown below. Black arrows indicate the naturally occurring HPFH mutations described at the HBG promoters, with the percentage of HbF expressed by heterozygous carriers^{8,17-20}. Mutations in the HBG1 and HBG2 promoters are indicated in grey and black, respectively. BCL11A (−118 to −113 nucleotides) and LRF (−203 to −194 nucleotides) binding sites (as described in ²) are highlighted. Arrows indicate the cleavage sites of the gRNAs employed in this study. (B-F) Globin expression analyses were performed in mature erythroblasts differentiated from Cas9-GFP⁺ HUDEP-2 erythroid progenitor cells. (B) Abundance of ^{G}γ+^{A}γ− and β-globin mRNAs, detected by RT-qPCR and expressed as percentage of (γ+β)-globins. (C) Representative flow cytometry plots showing the percentage of HbF⁺ cells. (D) Reverse-phase HPLC quantification of γ-, β- and δ-globin chains. β-like globin expression was normalized to α-globin. The ratio of a chains to non-α chains was unchanged in HBG-edited and control samples. (E) Representative cation-exchange HPLC chromatograms showing the different Hb types. The percentage of HbF tetramers over the total Hbs is indicated in brackets. (F) Quantification of HbF and acetylated HbF (HbF+AcHbF), HbA and HbA2 hemoglobin content by cation-exchange HPLC. We plotted the percentage of each Hb type over the total Hb tetramers. (G) ChIP-qPCR analysis of H3K27Ac in −197-edited HUDEP-2 cells and in control AAVS1-edited samples. ChIP was performed using an antibody against H3K27Ac and the corresponding control IgG. Results are shown as mean±SEM of three independent experiments. **** P≤0.0001, *** P≤0.001 and ** P≤0.01, * P≤0.05, ns P>0.05 by unpaired t-test.

FIG. 2. HbF up-regulation in SCD patient-derived RBCs upon Cas9/gRNA RNP delivery. (A) Percentage of InDels in mature erythroblasts derived from SCD HSPCs, as evaluated by TIDE. (B) Abundance of ^{G}γ+^{A}γ− and β-globin mRNAs, detected by RT-qPCR in primary mature erythroblasts and expressed as percentage of (γ+β3)-globins. Results were normalized to α-globin. Error bars denote standard deviation. (C) Flow cytometry plots showing the percentage of HbF⁺ cells and the median fluorescence intensity (MFI) (in brackets) in RBCs derived from control and HBG-edited SCD HSPCs. (D) Reverse-phase HPLC quantification of γ-, β- and δ-globin chains. β-like globin expression was normalized to α-globin. The ratio of α chains to non-α chains was unchanged in HBG-edited and control samples. (E) Quantification of HbF+AcHbF, HbA and HbA2 by cation-exchange HPLC. We plotted the percentage of each Hb type over the total Hb tetramers. (F) In vitro sickling assay measuring the proportion of sickled RBCs under hypoxic conditions (5 and 0% O₂) at different time points. We plotted the percentage of non-sickled cells.

FIG. 3. Deletion frequency at each nucleotide of the HBG promoters. A-C The analysis was performed in mature erythroblasts derived from adult SCD and CB healthy donor HSPCs. Location of LRF and BCL11A binding sites and the 13-bp HPFH deletion are indicated. Data are expressed as mean±SEM (n=3-4, 2-3 donors).

### EXAMPLE

Methods

Plasmid Construction

We used the CRISPOR webtool¹² to design gRNAs targeting the −200 and −158 HBG promoters. Double strand oligonucleotides containing the gRNA sequences were cloned into MA128 plasmid (provided by Dr. Mario Amendola, Genethon, France) using the BbsI restriction enzyme. The gRNA target sequences are listed below (PAM motif in bold).

| gRNA | Target sequence | |
|---|---|---|
| name | (5′ to 3′) | Strand |
| AAVS1 | GGGGCCACTAGGGACAGGATTGG | − |
| −197 | ATTGAGATAGTGTGGGGAAGGGG | + |
| −196 | CATTGAGATAGTGTGGGGAAGGG | + |
| −195 | GCATTGAGATAGTGTGGGGAAGG | + |
| −158 | TATCTGTCTGAAACGGTCCCTGG | − |
| −152 | CCATGGGTGGAGTTTAGCCAGGG | + |
| −151 | CCCATGGGTGGAGTTTAGCCAGG | + |
| −115 | CTTGTCAAGGCTATTGGTCAAGG | + |

Cell Line Culture

K562 were maintained in RPMI 1640 medium (Lonza) containing glutamine and supplemented with 10% fetal bovine serum (Lonza), HEPES (LifeTechnologies), sodium pyruvate (LifeTechnologies) and penicillin and streptomycin (LifeTechnologies). HUDEP-2¹³ cells were cultured and differentiated, as previously described¹⁴. Flow cytometry analysis of CD36, CD71 and GYPA expression and a standard May-Grumwald Giemsa staining were performed to evaluate the cell morphology.

Cell Line Transfection

K562 and HUDEP-2 cells were transfected with 4 μg of a Cas9-GFP expressing plasmid (pMJ920, Addgene) and 0.8 and 1.6 μg of gRNA-containing plasmid for K562 and HUDEP-2 transfections, respectively. We used AMAXA Cell Line Nucleofector Kit V (VCA-1003) for K562 and HUDEP-2 (U-16 and L-29 programs, respectively). GFP⁺ HUDEP-2 cells were sorted using SH800 Cell Sorter (Sony Biotechnology).

HSPC Purification and Culture

We obtained human cord blood CD34⁺ HSPCs from healthy donors. Human adult SCD CD34⁺ HSPCs were isolated from Plerixaflor mobilized SCD patients (NCT 02212535 clinical trial, Necker Hospital, Paris, France). Written informed consent was obtained from all adult subjects. All experiments were performed in accordance with the Declaration of Helsinki. The study was approved by the regional investigational review board (reference: DC 2014-2272, CPP Ile-de-France II “Hôpital Necker-Enfants malades”). HSPCs were purified by immuno-magnetic selection with AutoMACS (Miltenyi Biotec) after immunostaining with CD34 MicroBead Kit (Miltenyi Biotec).

48h prior to transfection, CD34⁺ cells (10⁶ cells/ml) were thawed and cultured in StemSpan (StemCell Technologies) supplemented with penicillin/streptomycin (Gibco) and the following recombinant human cytokines (Peprotech): 300 ng/mL SCF, 300 ng/mL Flt-3L, 100 ng/mL TPO and 60 ng/mL IL3, and StemRegenin1 at 250 nM (StemCell Technologies).

HSPC Transfection

The non-chemically-modified gRNA was composed of a tracrRNA (IDT) and a custom crRNA (IDT) assembled at 95° C. for 5 min in equimolar concentrations to produce a 180 μM duplex cr:tracrRNA guide. Chemically modified synthetic single gRNAs (sgRNAs) harboring 2′-O-methyl analogs and 3′-phosphorothioate non-hydrolysable linkages at the first three 5′ and 3′ nucleotides were resuspended at the concentration of 180 μM.

The cr:tracrRNA or sgRNAs were assembled at room temperature with a purified Cas9 protein at 90 μM (provided by Dr. Concordet) at a ratio 2:1 (gRNA:Cas9) to prepare ribonucleoprotein (RNP) complex. 200,000 human CD34⁺ cells were transfected with RNP particles using the P3 Primary Cell 4D-Nucleofector X Kit S (Lonza) and the CA137 program of the AMAXA 4D device (Lonza) with or without 90 μM or 180 μM transfection enhancer (IDT). After transfection, cells were plated at 50,000/mL in the erythroid differentiation medium. 18h after transfection, viability was measured by flow cytometry.

HSPC Differentiation

Transfected human HSPCs were differentiated to mature RBCs using a 3-step protocol¹⁵. From days 0 to 6, cells were grown in a basal erythroid medium supplemented with the following recombinant human cytokines: 100 ng/mL SCF (Peprotech), 5 ng/mL IL3 (Peprotech), and 3 IU/mL of EPO Eprex (Janssen-Cilag), and hydrocortisone (Sigma) at 10⁻⁶ M. From days 6 to 9, cells were cultured onto a layer of murine stromal MS-5 cells in basal erythroid medium supplemented only with 3 IU/mL EPO Eprex. Finally, from days 9 to 20, cells were cultured on a layer of MS-5 cells in basal erythroid medium but without cytokines.

Erythroid differentiation was monitored by May Grunwald-Giemsa staining, flow cytometry analysis of the erythroid surface markers CD36, CD71 and GYPA (CD36-V450, BD Horizon), CD71 (CD71-FITC, BD Pharmingen) and GYPA (CD235a-PECY7, BD Pharmingen). We used the nuclear dye DRAQ5 (eBioscience) to evaluate the proportion of enucleated RBCs. Flow cytometry analyses were performed using the Gallios analyzer and Kaluza software (Beckman-Coulter).

FACS Sorting of HSPC Populations

10⁶ healthy donor CB-derived CD34+ HSPCs were transfected as described above and plated at a concentration of 500,000/mL in StemSpan (StemCell Technologies) supplemented with penicillin/streptomycin (Gibco), 250 nM StemRegenin1 (StemCell Technologies) and the following recombinant human cytokines (Peprotech): 300 ng/mL SCF, 300 ng/mL Flt-3L, 100 ng/mL TPO and 60 ng/mL IL3. 18h after transfection, cells were stained with antibodies recognizing CD34 (CD34 PE-Cy7, 348811, BD Pharmingen), CD133 (CD133 PE, 130-113-748, Miltenyi Biotech) and CD90 (CD90 PE-Cy5, 348811, BD Pharmingen). Cells were sorted using FACSAria II (BD Biosciences). Sorted and unsorted populations were cultured at a concentration of 5×105/mL in cytokine-enriched medium (described above) for 4 days before collection for DNA extraction.

CFC Assay

The number of hematopoietic progenitors was evaluated by clonal colony-forming cell (CFC) assay. HSPCs were plated at a concentration of 1×10³ cells/mL in methylcellulose-containing medium (GFH4435, Stem Cell Technologies) under conditions supporting erythroid and granulo-monocytic differentiation. BFU-E and CFU-GM colonies were scored after 14 days. BFU-Es and CFU-GMs were randomly picked and collected as bulk populations (containing at least 25 colonies) or as individual colonies (35 to 45 colonies per sample) to evaluate genome editing efficiency.

PCR-Based Assays for Detection of Genome Editing Events

Genome editing was analyzed in HUDEP-2 cells at day 0 and day 9 of erythroid differentiation, and in cord blood and adult mobilized HSPC-derived erythroid cells at day 6 and day 14 of erythroid differentiation, respectively. Genomic DNA was extracted from control and edited cells using PURE LINK Genomic DNA Mini kit (LifeTechnologies) following manufacturer's instructions. To evaluate non-homologous end-joining (NHEJ) efficiency at gRNA target sites, we performed PCR followed by Sanger sequencing and TIDE analysis (Tracking of InDels by Decomposition)¹⁶.

InDels events were detected using the following primers:

| | | | Ampli- |
|---|---|---|---|
| Ampli- | | | con |
| fied | | | size |
| region | F/R | Sequence 5′-3′ | (bp) |
| HBG1 + | F | AAAAACGGCTGACAAAAGAAGTCCTGGTAT | 384 |
| HBG2 | | (SEQ ID NO: 5) | |
| promo- | R | ATAACCTCAGACGTTCCAGAAGCGAGTGTG | |
| ters | | (SEQ ID NO: 6) | |
| HBG1 | F | TACTGCGCTGAAACTGTGGC | 678 |
| promo- | | (SEQ ID NO: 7) | |
| ter | R | GGCGTCTGGACTAGGAGCTTATTG | |
| | | (SEQ ID NO: 8) | |
| HBG2 | F | GCACTGAAACTGTTGCTTTATAGGAT | 676 |
| promo- | | (SEQ ID NO: 9) | |
| ter | R | GGCGTCTGGACTAGGAGCTTATTG | |
| | | (SEQ ID NO: 10) | |
| AAVS1 | F | CAGCACCAGGATCAGTGAAA | 481 |
| | | (SEQ ID NO: 11) | |
| | R | CTATGTCCACTTCAGGACAGCA | |
| | | (SEQ ID NO: 12) | |

F, forward primer; R, reverse primer.

RT-qPCR Analysis of Globin Transcripts

Total RNA was extracted from differentiated HUDEP-2 (day 9) and in primary mature SCD erythroblasts (day 13) using RNeasy micro kit (QIAGEN) following manufacturer's instructions. Mature transcripts were reverse-transcribed using SuperScript First-Strand Synthesis System for RT-qPCR (Invitrogen) with oligo (dT) primers. RT-qPCR was performed using iTaq universal SYBR Green master mix (Biorad). RT-qPCR plates were run on Viia7 Real-Time PCR system (ThermoFisher Scientific). Primer sequences used for RT-qPCR are listed below.

| | | |
|---|---|---|
| HBA | F | 5′-CGGTCAACTTCAAGCTCCTAA-3′ |
| | | (SEQ ID NO: 13) |
| | R | 5′-ACAGAAGCCAGGAACTTGTC-3′ |
| | | (SEQ ID NO: 14) |
| HBB | F | 5′-GCAAGGTGAACGTGGATGAAGT-3′ |
| | | (SEQ ID NO: 15) |
| | R | 5′-TAACAGCATCAGGAGTGGACAGA-3′ |
| | | (SEQ ID NO: 16) |
| HBG1 + | F | 5′-CCTGTCCTCTGCCTCTGCC-3′ |
| HBG2 | | (SEQ ID NO: 17) |
| | R | 5′-GGATTGCCAAAACGGTCAC-3′ |
| | | (SEQ ID NO: 18) |
| HBD | F | 5′-CAAGGGCACTTTTTCTCAG-3′ |
| | | (SEQ ID NO: 19) |
| | R | 5′-AATTCCTTGCCAAAGTTGC-3′ |
| | | (SEQ ID NO: 20) |

F, forward primer; R, reverse primer.

Reverse Phase (RP) HPLC Analysis of Globin Chains

RP-HPLC analysis was performed using a NexeraX2 SIL-30AC chromatograph (Shimadzu) and the LC Solution software. Globin chains were separated by HPLC using a 250×4.6 mm, 3.6 μm Aeris Widepore column (Phenomenex). Samples were eluted with a gradient mixture of solution A (water/acetonitrile/trifluoroacetic acid, 95:5:0.1) and solution B (water/acetonitrile/trifluoroacetic acid, 5:95:0.1). The absorbance was measured at 220 nm.

Cation-Exchange HPLC Analysis of Hemoglobin Tetramers

Cation-exchange HPLC analysis was performed using a NexeraX2 SIL-30AC chromatograph (Shimadzu) and the LC Solution software. Hemoglobin tetramers were separated by HPLC using a 2 cation-exchange column (PolyCAT A, PolyLC, Coulmbia, Md.). Samples were eluted with a gradient mixture of solution A (20 mM bis Tris, 2 mM KCN, pH=6.5) and solution B (20 mM bis Tris, 2 mM KCN, 250 mM NaCl, pH=6.8). The absorbance was measured at 415 nm.

Flow Cytometry Analysis

We labeled HUDEP-2 and HSPC-derived RBCs with antibodies against CD36 (CD36-V450, BD Horizon), CD71 (CD71-FITC, BD Pharmingen) and CD235a (CD235a-APC, BD Pharmingen; CD235a-PECY7, BD Pharmingen) surface markers. Differentiated HUDEP-2 and HSPC-derived RBCs were fixed and permeabilized using BD Cytofix/Cytoperm solution (BD Pharmingen) and stained with antibodies recognizing HbF (HbF-APC, MHF05, Life Technologies and HbF FITC, 552829, BD Pharmingen). We performed flow cytometry analyses using Fortessa X20 flow cytometer (BD Biosciences) and Gallios (Beckman Coulter).

Chromatin Immunoprecipitation (ChIP) Assay

After 5 days of differentiation, −197 and AAVS1 HUDEP-2 bulk populations were collected for ChIP assays. ChIP experiments were performed as previously described⁶. Briefly, chromatin was crosslinked for 10 minutes at room temperature with 1% formaldehyde-containing medium. Nuclear extracts were sonicated using the Bioruptor Pico Sonication System (Diagenode). The equivalent of 2 million cells crosslinked DNA was pulled down at 4° C. overnight using an antibody (1 μg per million cells) against H3K27Ac (ab4729, Abcam) or control IgG (Rabbit, sc-2025, Santa Cruz). Chromatin crosslinking was then reversed at 65° C. for at least 4 hours and DNA was purified (QIAquick PCR purification kit, QIAGEN). Quality check of the fragments generated was carried out using the Agilent Bioanalyzer. We used quantitative SYBR Green PCR (Applied Biosystems) to evaluate H3K27Ac at different genomic loci. qPCR experiments were performed on Viia7 Real-Time PCR system (ThermoFisher Scientific). Primers are listed below.

| | | |
|---|---|---|
| HBB | F | 5′-TGCTCCTGGGAGTAGATTGG-3′ |
| | | (SEQ ID NO: 21) |
| | R | 5′-TGGTATGGGGCCAAGAGATA-3′ |
| | | (SEQ ID NO: 22) |
| HBG | F | 5′-ACAAGCCTGTGGGGCAAGGTG-3′ |
| | | (SEQ ID NO: 23) |
| | R | 5′-GCCAGGCACAGGGTCCTTCC-3′ |
| | | (SEQ ID NO: 24) |

F, forward primer; R, reverse primer.

Sickling Assay

In vitro-generated SCD RBCs were exposed to an oxygen-deprived atmosphere (5 and 0% O₂), and the time course of sickling was monitored in real time by video microscopy, capturing images every 20 minutes using the AxioObserver Z1 microscope (Zeiss) and a 20× objective. Images of the same fields were taken throughout all stages and processed with ImageJ to determine the percentage of non-sickled RBCs per field of acquisition in the total RBC population.

Statistics

All statistical analyses were performed using Unpaired t tests with Prism4 software (GraphPad). The threshold for statistical significance was set to P<0.05.

Results

### Example 1

Targeting Multiple Regions in the HBG Promoters Induces HbF Expression in Adult HUDEP-2 Erythroid Cells

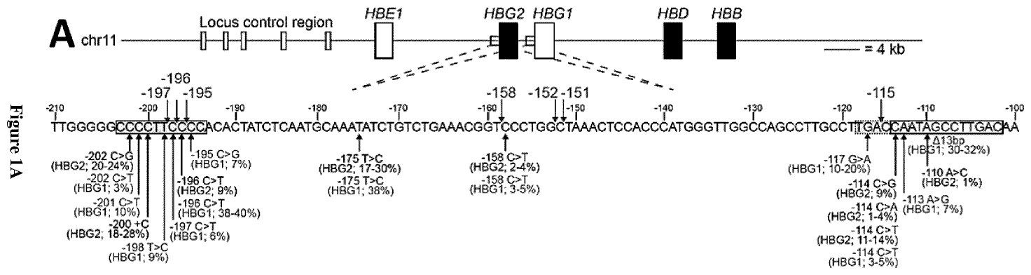

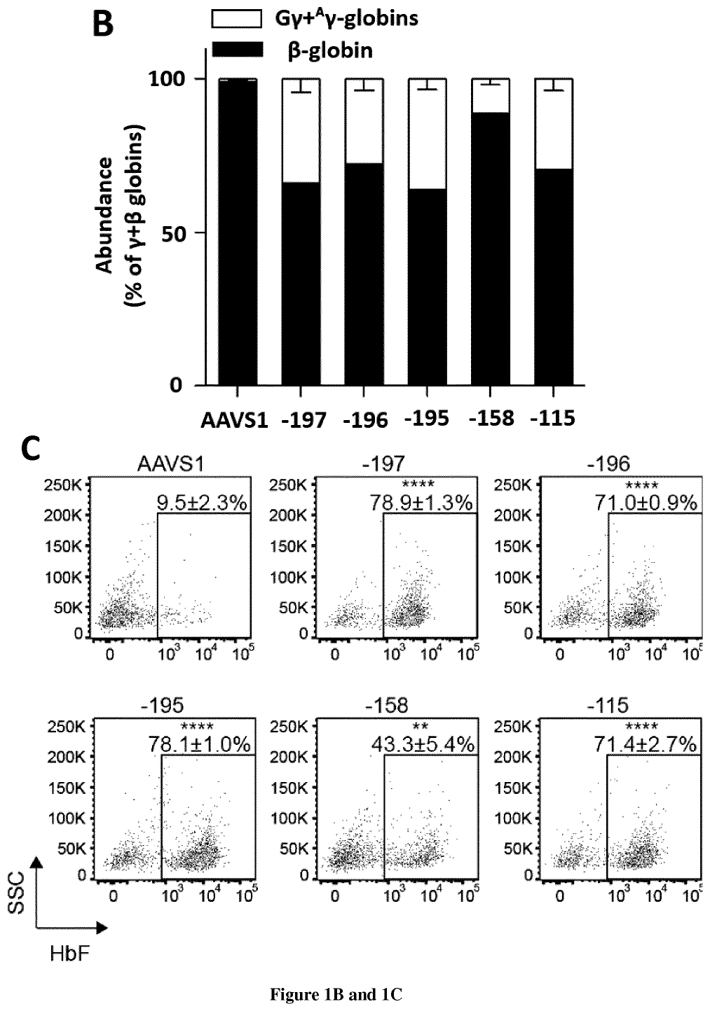

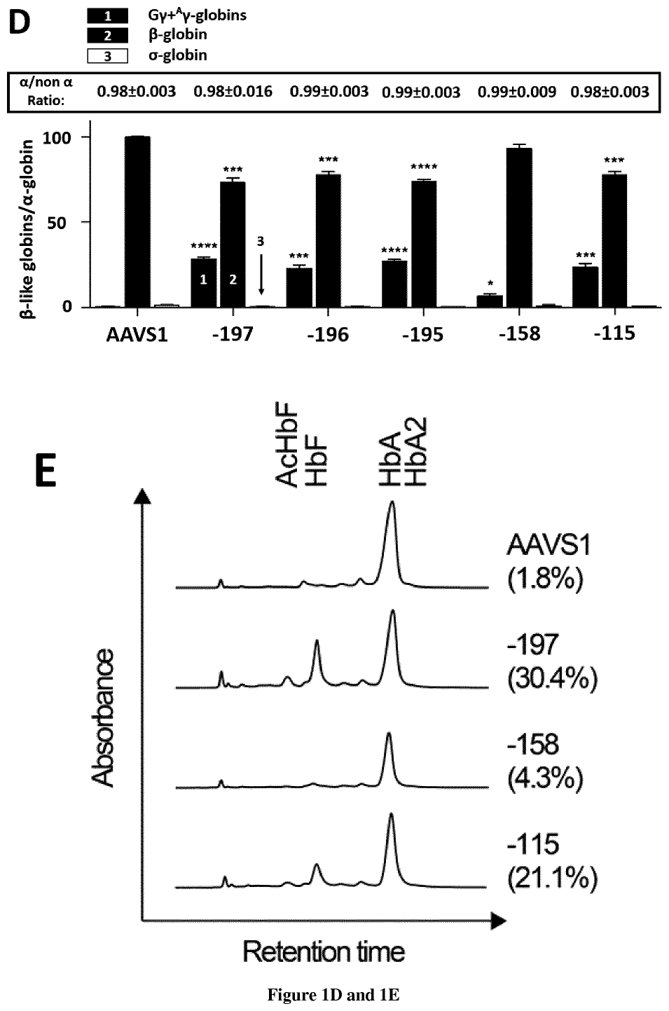

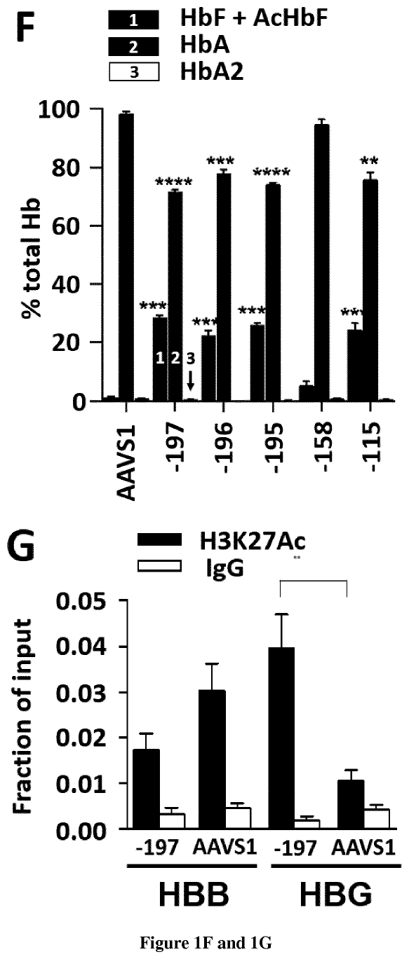

HPFH mutations and SNPs associated with high HbF levels have been described in multiple regions of the HBG promoters (−200, −158 and −115; FIG. 1A). HPFH mutations in the −200 and −115 regions alter the binding of transcriptional repressors². We hypothesized that disruption of these regions via CRISPR/Cas9 could potentially lead to HbF de-repression. We designed guide RNAs (gRNAs) binding the −200 (−197, −196 and −195) binding site of the HbF repressor LRF and the −158 region (−158, −152 and −151). In parallel, we used a gRNA targeting the −115 region and leading to HbF reactivation in adult HUDEP-2 erythroid cell line and HSPC-derived RBCs⁷, likely by disrupting a binding site for the HbF repressor BCL11A² (FIG. 1A). Plasmid delivery of individual gRNAs and a Cas9-GFP fusion in fetal erythroleukemia cell line K562 revealed a similar editing efficiency for the three gRNAs targeting the −200 region, whereas gRNA −158 displayed the highest editing efficiency at the −158 region. High cleavage efficiency was also observed for the −115 gRNA and the control gRNA targeting the AAVS1 locus (data not shown).

We next employed the adult HUDEP-2 cell line, expressing low levels of HbF, to evaluate HbF de-repression following disruption of the −200, −158 and −115 nt regions upstream of the HBG TSSs. Of note, sequencing of the HBG promoters in HUDEP-2 cells revealed the presence of a −158 T>C heterozygous SNP in the HBG2 promoter (data not shown).

After plasmid transfection, Cas9-GFP⁺ HUDEP-2 cells were sorted and differentiated in mature erythroblasts. The editing rate was similar at day 0 and day 9 of erythroid differentiation, showing no counter-selection of genome edited cells during erythroid maturation (data not shown). Overall, the genome editing efficiency in cells differentiated from Cas9-GFP⁺ HUDEP-2 was >77% for all samples, with the exception of −158 gRNA, whose cleavage efficiency was 50%±4% (data not shown). Of note, using this gRNA we obtained a significantly higher editing frequency at the HBG1 promoter, compared to the HBG2 promoter (68%±1% vs 40%±6%; data not shown). The presence of a −158 T>C heterozygous SNP in the HBG2 promoter likely reduce the binding of the −158 gRNA recognizing the wild-type promoter and therefore that could contribute to the overall lower disruption efficiency at the −158 site. Similar editing rates at HBG2 and HBG1 promoters were obtained with the other gRNAs (data not shown). As expected⁷, one third of InDels generated using the −115 gRNA were 13-nt deletions (data not shown).

Deep sequencing of PCR-amplified HBG promoters in −197-edited samples allowed a quantitative detection of the mutations generated upon delivery of the CRISPR/Cas9 system. The most prevalent InDels were 4-nt [CCCC], 6-nt [TTCCCC] and 2-nt [CC] deletions and 1-nt [C] insertion. Importantly, the LRF binding site described by Martyn and colleagues², was disrupted in all the alleles (data not shown).

Editing of the HBG promoters did not alter the erythroid cell expansion (data not shown) or differentiation, as assessed by morphological analysis (data not shown) and flow cytometry analysis of GYPA, CD71 and CD36 erythroid markers (data not shown). Concordantly, in silico evaluation of the putative off-target activity of all the gRNAs revealed that none of the predicted off-targets fall within coding regions of genes involved in RBC maturation or physiology (data not shown).

γ-globin expression was then assessed in control and HBG-edited differentiated HUDEP-2 cells. Disruption of the −200 region led to an increased production of γ-globin transcripts, paralleled by a decreased synthesis of adult β-globin and δ-globin mRNAs (FIG. 1B). Similar changes in globin expression were observed upon targeting of the −115 region (FIG. 1B). A lower γ-globin reactivation was observed upon targeting of the −158 region, which was consistent with the lower cleavage efficiency of the −158 gRNA in HUDEP-2 cells (FIG. 1B).

Flow cytometry analysis of the differentiated HUDEP-2 samples targeted with the −197, −196 and −195 gRNAs revealed almost pancellular HbF expression (79%±1%, 71%±1% and 78%±1% of cells expressing HbF, respectively [F-cells], P<0.0001 compared to AAVS1 controls). Similar results were obtained upon targeting of the −115 region (71%±3%, P<0.0001), and an average of 43%±5% (P=0.005) of F-cells were observed in the −158-edited samples (FIG. 1C).

Reverse-phase HPLC showed that disruption of the −200 region led to the highest γ-globin chain levels. ^{G}γ- and ^{A}γ-globin chains accounted for up to 28%±1% of α-globin chain in samples edited with the −197 gRNA, showing a highly significant increase from the basal γ-globin chain levels detected in the AAVS1 control sample (0.3%±0.3%, P<0.0001) (FIG. 1D). Genome editing of the HBG promoters at the −115 and −158 regions respectively led to 24%±2% (P=0.0004) and 7%±2% (P=0.015) of γ-globin chains, compared to control cells (FIG. 1D). For all edited samples, we noted that the ^{A}γ-chain levels were higher compared to ^{G}γ-chains (data not shown). Additionally, and similarly to the changes observed at mRNA level, β-globin polypeptide synthesis significantly decreased in samples targeted with the −197, −196 and −195 gRNAs and with the −115 gRNA (FIG. 1D). We noted no change in δ-globin chain levels upon editing of the HBG promoters (FIG. 1D). Importantly, HBG-edited HUDEP-2 showed a normal ratio of α to non-α chains, indicating that the reduction in β-globin expression is well compensated by the reactivation of γ-globin synthesis (FIG. 1D). Finally, HbF tetramer production was quantified by cation exchange HPLC (FIGS. 1E and F). Samples edited with the −197 and −195 gRNAs displayed the highest HbF levels, with HbF representing 28%±1% and 26%±1% of the hemoglobin tetramers, compared to control cells (1%±1%, P<0.0001). Genome editing using the −196 or −115 gRNAs led also to a significant reactivation of HbF, representing respectively 22%±1% and 24%±3% of total hemoglobins (P<0.001). For −158-edited samples, HbF accounted for 5%±2% of the hemoglobin tetramers (P<0.05 compared to control cells, FIG. 1F). Concomitantly, in cells displaying high HbF levels, HbA expression decreased significantly, compared to the AAVS1 control samples. Upon HBG-promoter editing, the levels of HbA2 remained stable (FIG. 1F).

To assess the epigenetic modifications induced by the disruption of LRF binding site, we performed ChIP experiments for H3K27 acetylation (H3K27Ac), a chromatin mark associated with active regulatory regions, in −197 and in control AAVS1 HUDEP-2 cells. We detected higher H3K27Ac at the HBG genes in −197 edited cells compared to controls (FIG. 1G). Concomitantly, H3K27Ac tends to be reduced at the HBB gene in −197 edited cells compared to controls (FIG. 1G). These results suggest that impaired LRF binding at the −200 region blocks the recruitment of the LRF-interacting Nucleosome Remodeling Deacetylase (NuRD) complex, which contains HDAC1 and 2 histone deacetylases that maintain a closed chromatin conformation⁶.

Targeting of the HBG Promoters Up-Regulates HbF in SCD Patient-Derived RBCs

As plasmid delivery in primary cells is associated with high cell toxicity, we developed a ribonucleoprotein (RNP)-based, selection free strategy to efficiently edit the HBG promoters in HSPCs with a minimal impact on the cell viability. To this purpose, we assembled Cas9 protein with the −197 gRNAs with or without 2′-O-methyl analogs and 3′-phosphorothioate non-hydrolysable linkages at the first three 5′ and 3′ nucleotides. Delivery of RNP complexes containing Cas9 and the chemically modified −197 gRNA in cord blood-derived HSPCs increased the genome editing efficiency from 11% to 32%, as compared to the delivery of the non-modified −197 gRNA (data not shown). Genome editing efficiency was further improved by the addition of a transfection enhancer oligonucleotide, at the doses of 90 μM and 180 μM. Similar results were obtained with the −196 and −195 chemically modified gRNAs. The highest genome editing frequency was achieved with the combination of chemically modified gRNAs and 90 μM of the enhancer, leading to a cleavage efficiency of 80%, 72% and 84% with the −197, −196 and −195 gRNAs, respectively (data not shown). A good cell viability was observed using these transfection conditions, as compared to the untransfected control (data not shown).

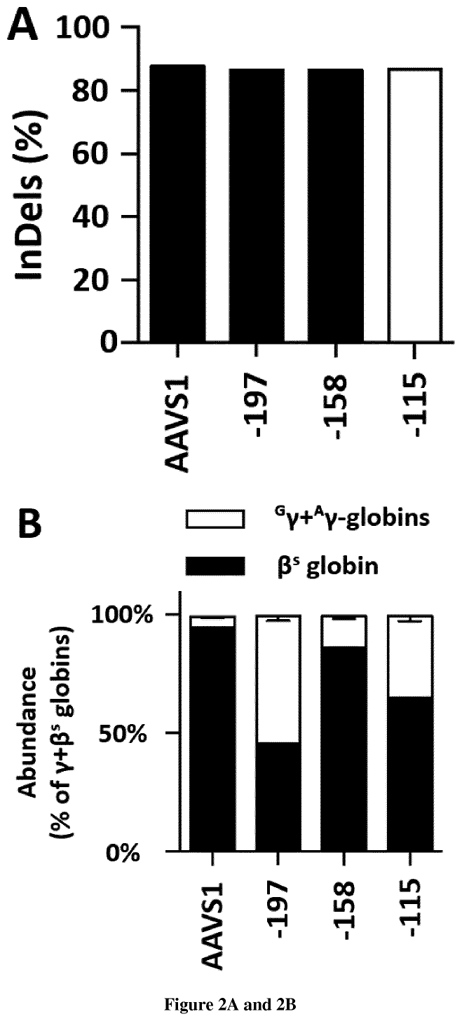

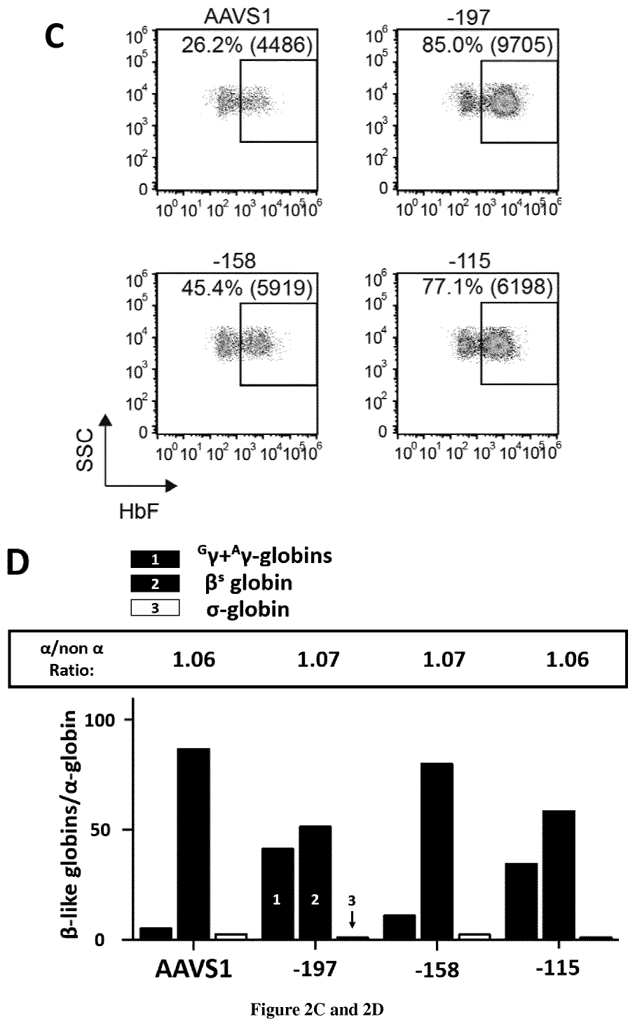

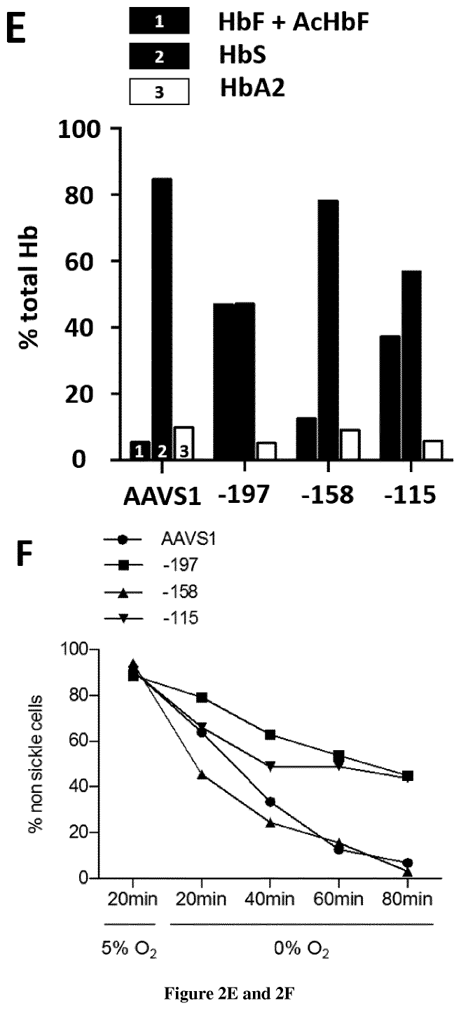

Plerixafor-mobilized SCD HSPCs were transfected with RNP particles containing the −197, −158 or −115 gRNAs targeting the HBG promoters or the control AVVS1 gRNA. Following erythroid expansion, the genome editing efficiency was assessed in mature erythroblasts, and reached 87-88% in all the edited samples (FIG. 2A). To evaluate the HbF reactivation and the correction of the SCD cell phenotype following the editing of HBG promoters, cells were terminally differentiated to enucleated RBCs. Importantly, editing of the HBG promoters did not affect the erythroid differentiation, as evaluated by flow cytometry analysis of stage-specific erythroid markers (data not shown), by morphological analysis (data not shown), and by assessment of the enucleation rate, determined as the percentage of cells negative for the nuclear dye DRAQ5 (data not shown).

In HBG-edited primary erythroblasts, qRT-PCR analysis showed an increase in γ-globin expression, which was more pronounced in the −197 sample (˜10-fold) where γ-globin mRNA accounted for ˜50% of the total β-like globin mRNAs (FIG. 2B). A parallel reduction in β^{S}- and δ-globin mRNA levels was observed in −197 and −115 samples (data not shown). Editing with the −197 gRNA induced a marked increase in the proportion of F-cells, rising to 85%, from a basal level of 26% in control cells, along with a pronounced increase in the HbF content (MFI) (FIG. 2C). RBCs derived from HSPCs edited with the −158 and −115 gRNAs displayed 45% and 77% of F-cells, respectively and an increased HbF content, although lower compared to the −197 sample (FIG. 2C). HPLC analyses confirmed the potent HbF reactivation in the −197 gRNA sample, with mutant β^{S}-globin and HbS levels comparable to a healthy heterozygous SCD carrier (FIGS. 2D and 2E). ^{A}γ- and ^{G}γ-chain levels were similar in all edited samples except for the −115, where ^{A}γ-globins were more abundant than ^{G}γ-chains (data not shown).

To assess the effect of the HbF reactivation induced by the editing of the HBG promoters, we performed an in vitro deoxygenation assay inducing the sickling of RBCs derived from patient HSPCs. Upon deoxygenation, the majority of the control RBCs rapidly acquired a sickle morphology (FIG. 2F). A similar phenotype was observed in the sample edited with the −158 gRNA (FIG. 2F). In contrast, editing at −197 and −115 led to a robust correction of the SCD cell phenotype, which was more pronounced in the 197 samples at earlier, likely more physiological time points (FIG. 2F).

### Example 2

We then compared the activity of 3 gRNAs targeting the LRF binding site in CD34⁺ HSPCs obtained from SCD patients by plerixafor mobilization. SCD HSPCs were transfected with RNP complexes containing either the gRNAs targeting the HBG promoters or the control AAVS1 gRNA. Following erythroid differentiation, genome editing efficiency in mature erythroblasts achieved values of ≥80% in cells transfected with −197, −196, −195 and −115 gRNAs (data not shown). Editing frequency with the −158 gRNA was variable because of the presence of the C>T SNP at that position in a fraction of the SCD donors (data not shown). Genome editing efficiency was similar between the HBG2 and HBG1 promoters except for samples harboring the −158 SNP and treated with the −158 gRNA (data not shown).

Control and edited SCD HSPCs were plated in clonogenic cultures (colony forming cell [CFC] assay) allowing the growth of erythroid (BFU-E) and granulomonocytic (CFU-GM) progenitors. Genome editing efficiency was comparable in pooled BFU-Es and CFU-GMs that showed a similar InDel profile (data not shown). Clonal analysis of single CFCs revealed that >85% of hematopoietic progenitors were edited at the target sites, with ˜86% and ˜67% of BFU-Es and CFU-GMs, respectively, displaying ≥3 edited HBG promoters (data not shown). Transfection with the full RNP complex reduced the number of hematopoietic progenitors by 10 to 50% compared to transfection of Cas9 protein alone (data not shown).

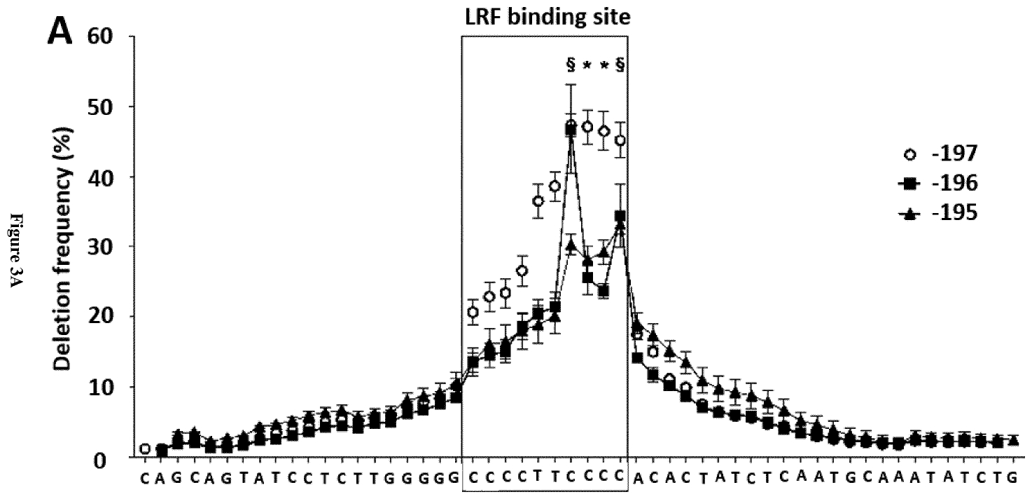

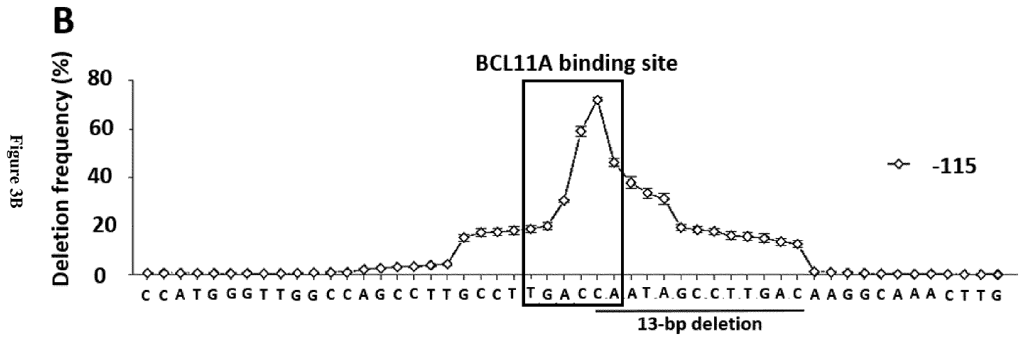

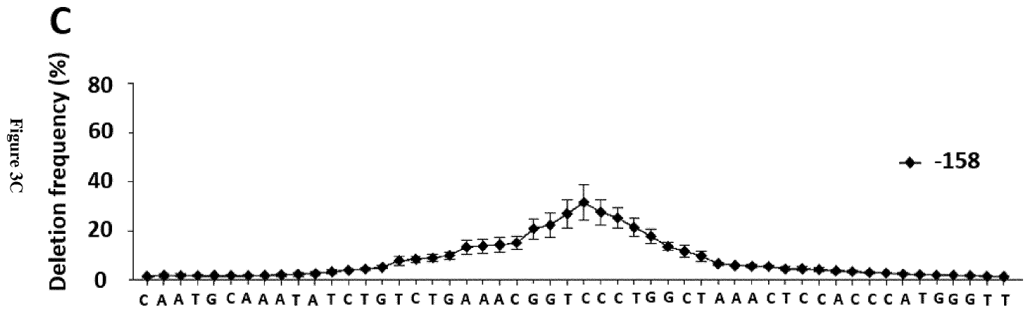

Previous reports suggested that HSCs, the target of therapeutic genome editing, are preferentially edited via the NHEJ mechanism^{39,40}. On the contrary, MMEJ repair pathway, which takes place through annealing of short stretches of identical sequence flanking the double-strand break (DSB), may be less active^{39,40}. Therefore, for each gRNA we evaluated the frequency of mutations with or without microhomology (MH)-motifs as a proxy for the relative contribution of MMEJ- and NHEJ-mediated events. Amongst the editing events, deletions were predominant, and a variable fraction of them (30% to 50%) was associated with the presence of MH-motifs in the target sequence (data not shown).). In particular, MMEJ events at the LRF binding site were caused by the presence of two stretches of 4 cytidines (FIG. 1A). Amongst the total InDels, the frequency of events associated with MH-motifs was significantly higher for the −197 (38±3%) and −195 (32±1%) gRNAs than for the −196 gRNA (23±1%). The gRNAs targeting the LRF binding site induced distinct InDel profiles: −196 and −195-edited cells harbored mainly 1-bp insertions and 1-2-bp deletions, while the −197 gRNA generated the largest fraction of >2-bp deletion events, of which ˜45% were associated with MH-motifs (data not shown). Importantly, virtually all the editing events generated by the −197, −196 and −195 gRNAs disrupted the LRF binding site (data not shown). Of note, the proportion of nucleotides in the LRF binding site that were lost as a result of editing was higher in −197 than in −196 and −195 samples (FIG. 3A-C). As expected, the −115 gRNA caused disruption of the BCL11A binding site 14. In these cells, MMEJ-mediated events were mainly 13-bp deletions partially spanning the BCL11A binding site (FIG. 3A-C). Finally, the −158 gRNA generated mostly 1-bp insertions and small deletions around the cleavage site (FIG. 3A-C). To evaluate CRISPR/Cas9-mediated genetic modification of the CD34+ cell fraction containing bona fide HSCs, HBG-promoter editing was assessed in FACS-isolated HSPC subpopulations 18, after transfection of the −197 and −196 gRNAs, associated with high and low frequencies of deletions associated with MH-motifs, respectively. Editing frequencies were comparable between primitive CD34+/CD133+/CD90+ and early CD34+/CD133+/CD90− progenitors and between CD34+/CD133− committed progenitors and unsorted CD34+ cells even in case of a limited genome editing efficiency, with a similar InDel profile across the different CD34+ cells subpopulations (data not shown). It is noteworthy that deletions likely generated via MMEJ occurred even in the more primitive, HSC-enriched populations (data not shown). All together, these results suggest that the LRF binding site can be efficiently targeted in primitive progenitors and potentially in bona fide HSCs.

To evaluate HbF reactivation and correction of the SCD cell phenotype upon HBG promoter editing, SCD HSPCs were terminally differentiated into enucleated RBCs. Editing of the HBG promoters did not affect erythroid differentiation, as evaluated by flow cytometry analysis of stage-specific erythroid markers and RBC enucleation, and by morphological analysis (data not shown). Editing of the −200 region led to increased levels of γ-globin mRNAs, which accounted for 48±3% of total β-like globin transcripts in cells transfected with the −197 gRNA (data not shown). The proportion of F-cells in cells transfected with the −197, −196 and −195 gRNAs was 81±1%, 74±2% and 74±2%, respectively (data not shown). Analysis of −197- and −196-edited erythroblasts sorted by cytofluorimetry based on the intensity of HbF expression revealed a positive correlation between InDel frequency and extent of γ-globin production, indicating that the efficacy of HbF reactivation likely increases when targeting a higher number of HBG promoters per cell (data not shown). Editing of the −115 region led to HBG de-repression and a proportion of 80±2% of F-cells, while γ-globin reactivation was less pronounced in the −158 samples (55±5% of F-cells, data not shown). It is noteworthy that for the −158 gRNA, HBG de-repression was still modest in RBCs derived from HSPCs harboring >85% of edited HBG promoters (data not shown), indicating that the −158 region contains a sequence that modestly contributes to inhibition of γ-globin expression in adult cells. This is consistent with the mild increase in HbF known to be associated with the −158 SNPs. RP-HPLC showed a significant increase in γ-globin chain expression and a reciprocal reduction in βS-globin levels in the RBC progeny of −200 and −115 edited HSPCs, with no evidence of imbalance in the α/non-α globin chain synthesis (data not shown). In −197-edited cells, the increase of γ-globin chains and the reduction of βS-globin levels resulted in an inversion of the β/γ globin ratio. Comparable Aγ- and Gγ-globin levels were detected in most of the samples analyzed. However, in −115-edited cells, HbF was mainly composed of Aγ-globin (data not shown). CE-HPLC confirmed that editing of the −200 region produced an Hb profile comparable to asymptomatic heterozygous carriers, with HbF representing up to 47±3% of the total Hb tetramers (−197 samples; data not shown).

To assess the effect of HbF reactivation on the sickling phenotype, we performed an in vitro deoxygenation assay that induces sickling of RBCs under hypoxia. At an oxygen concentration of 0%, ˜80% of control SCD RBCs acquired a sickled shape (data not shown). Targeting of the −158 region essentially failed to rescue the SCD phenotype (29±13% of non-sickling RBCs; data not shown). In −115-edited samples, HbF reactivation prevented the sickling of 56±9% of RBCs (data not shown). Interestingly, a marked correction of the SCD phenotype was achieved upon disruption of the LRF binding site, with 69±6% (−196) to 79±7% (−197) of cells that maintained a biconcave shape under hypoxia (data not shown).

Importantly, even gRNAs generating predominantly 1-2-bp InDels (−195 and −196) induced γ-globin levels that were sufficient to inhibit sickling in a large fraction of RBCS. These results show that editing of the repressor binding sites in the HBG promoters leads to reactivation of HbF sufficient to revert the sickling phenotypes in erythrocytes differentiated from CD34+ HSPCs derived from SCD patients.

Discussion

Allogeneic HSC transplantation is the only definitive cure for patients affected by β-thalassemia or SCD. Transplantation of autologous, genetically modified HSCs represents a promising therapeutic option for patient lacking a compatible HSC donor²¹. Compared with current lentiviral-based gene addition approaches, therapeutic strategies aimed at forcing a β-to-γ-globin switch have the advantage of guaranteeing high-level expression of the endogenous γ-globin genes and, in the case of SCD, reduction of the β^{S}-globin synthesis. Knockdown of the transcriptional repressor LRF increases HbF expression but delays erythroid differentiation⁶ and therefore is not a safe therapeutic approach. Here, instead of targeting the expression of a transcription factor essential for erythropoiesis, we used a CRISPR/Cas9 strategy to disrupt the cis-regulatory element involved in LRF-mediated fetal globin silencing and mimic the effect of HPFH mutations. By using three different gRNAs targeting the LRF binding site, we achieved a robust, virtually pancellular HbF reactivation and a concomitant reduction in βS-globin levels, recapitulating the phenotype of asymptomatic SCD-HPFH patients^{22,23}. Notably, a proportion of HbF>30% in 70% of RBCs has been proposed as the minimal requirement to inhibit HbS polymerization and mitigate the clinical SCD manifestations²³. In some edited samples, HbF levels exceeded 40% of total Hb, suggesting that CRISPR/Cas9 mediated disruption of the LRF binding site is even more potent than naturally occurring HPFH point mutations in reactivating HbF expression. RBCs derived from edited HSPCs displayed HbF levels sufficient to significantly ameliorate the SCD cell phenotype. It is noteworthy that this approach can potentially be applied also to β-thalassemias, where elevated fetal γ-globin levels could compensate for β-globin deficiency.

The development of a selection-free, optimized editing protocol allowed us to obtain a high editing frequency at the LRF binding site in primary human HSPCs and in HSC-enriched cell populations, which, unexpectedly, showed both NHEJ and MMEJ-mediated events. However, similarly to the homology-directed repair (HDR) mechanism¹¹ (used to correct disease-causing mutations^{24, 25, 26}), MMEJ repair pathway occurs in actively dividing cells²⁷. Therefore, we cannot exclude that MMEJ might not be efficient in the quiescent long-term repopulating HSCs^{39, 40}. Xenotransplantation of HSPCs edited using the gRNAs targeting the LRF binding site in immunodeficient mice^{28,29} will allow to assess the editing in long-term repopulating HSCs and the extent of HbF reactivation in their RBC progeny. However, it is noteworthy that even short InDels generated mainly by NHEJ (e.g., −196 gRNA) were productive in terms of HbF de-repression and correction of the SCD cell phenotype, thus showing that this strategy could be effective in bona fide HSCs.

Should the observed editing frequency be confirmed in long-term repopulating HSCs, this approach would guarantee the efficiency required to achieve clinical benefit in SCD and β-thalassemia. Importantly, the clinical history of allogeneic HSC transplantation for both diseases suggests that a limited fraction of genetically corrected HSCs would be sufficient to achieve a therapeutic benefit given the in vivo selective survival of corrected RBCs or erythroid precursors³⁰⁻³⁵. The minimal fraction of genetically modified HSCs would likely depend on the extent of fetal γ-globin expression that could confer a survival advantage to erythroid precursors and mature RBCs^{21,22,36}. In particular, since our approach generates a heterozygous phenotype, studies on mixed chimerism in SCD patients transplanted with HSCs from a SCD carrier suggest that an HSC genetic modification rate ≥30% would be sufficient to improve the SCD clinical phenotype^{32, 34, 35}.

Importantly, disrupting either the LRF or the BCL11A binding site in the HBG promoters induced significant HbF production. Given the independent role of LRF and BCL11A in γ-globin repression⁶, combined strategies aimed at evicting simultaneously both repressors from the γ-globin promoters could have an additive effect on HbF reactivation. Albeit a Cas9-nuclease-based strategy targeting both the −115 and the −200 regions would probably trigger the deletion of the −115-to-200 intervening sequence (that would be detrimental for promoter activity¹⁷), this study paves the way for the use of novel DSB-free editing strategies (e.g., base-editing³⁸) to simultaneously disrupt both LRF and BCL11A repressor binding sites in the γ-globin promoters.

Overall, our study provides proof of concept for a novel approach to treat SCD by targeting a repressor binding site in the γ-globin promoters to induce de-repression of fetal hemoglobin and a concomitant decrease in HbS synthesis. The same approach could be beneficial also in the case of β-thalassemia, providing a less complex and more economical gene therapy approach compared to the use of lentiviral vectors to deliver a functional β-globin gene.

### REFERENCES

Throughout this application, various references describe the state of the art to which this invention pertains. The disclosures of these references are hereby incorporated by reference into the present disclosure.
1. Forget, B. G. Molecular basis of hereditary persistence of fetal hemoglobin. Ann. N. Y. Acad. Sci. 850, 38-44 (1998).
2. Martyn, G. E. et al. Natural regulatory mutations elevate the fetal globin gene via disruption of BCL11A or ZBTB7A binding. Nat. Genet. 50, 498-503 (2018).
3. Wienert, B. et al. KLF1 drives the expression of fetal hemoglobin in British HPFH. Blood 130, 803-807 (2017).
4. Wienert, B. et al. Editing the genome to introduce a beneficial naturally occurring mutation associated with increased fetal globin. Nat. Commun. 6, 7085 (2015).
5. Liu, N. et al. Direct Promoter Repression by BCL11A Controls the Fetal to Adult Hemoglobin Switch. Cell 173, 430-442.e17 (2018).
6. Masuda, T. et al. Transcription factors LRF and BCL11A independently repress expression of fetal hemoglobin. Science 351, 285-289 (2016).
7. Traxler, E. A. et al. A genome-editing strategy to treat β-hemoglobinopathies that recapitulates a mutation associated with a benign genetic condition. Nat. Med. 22, 987-990 (2016).
8. Gilman, J. G. & Huisman, T. H. DNA sequence variation associated with elevated fetal G gamma globin production. Blood 66, 783-787 (1985).
9. Labie, D. et al. The −158 site 5′ to the G gamma gene and G gamma expression. Blood 66, 1463-1465 (1985).
10. Galarneau, G. et al. Fine-mapping at three loci known to affect fetal hemoglobin levels explains additional genetic variation. Nat. Genet. 42, 1049-1051 (2010).
11. Genovese, P. et al. Targeted genome editing in human repopulating haematopoietic stem cells. Nature 510, 235-240 (2014).
12. Haeussler, M. et al. Evaluation of off-target and on-target scoring algorithms and integration into the guide RNA selection tool CRISPOR. Genome Biol. 17, 148 (2016).
13. Kurita, R. et al. Establishment of immortalized human erythroid progenitor cell lines able to produce enucleated red blood cells. PloS One 8, e59890 (2013).
14. Canver, M. C. et al. BCL11A enhancer dissection by Cas9-mediated in situ saturating mutagenesis. Nature 527, 192-197 (2015).
15. Giarratana, M.-C. et al. Ex vivo generation of fully mature human red blood cells from hematopoietic stem cells. Nat. Biotechnol. 23, 69-74 (2005).
16. Brinkman, E. K., Chen, T., Amendola, M. & van Steensel, B. Easy quantitative assessment of genome editing by sequence trace decomposition. Nucleic Acids Res. 42, e168 (2014).
17. Martyn, G. E., Quinlan, K. G. R. & Crossley, M. The regulation of human globin promoters by CCAAT box elements and the recruitment of NF-Y. Biochim. Biophys. Acta 1860, 525-536 (2017).
18. Pissard, S., M'rad, A., Beuzard, Y. & Romeo, P. H. A new type of hereditary persistence of fetal haemoglobin (HPFH): HPFH Tunisia beta+(+C-200)G gamma. Br. J. Haematol. 95, 67-72 (1996).
19. Patrinos, G. P., Kollia, P., Loutradi-Anagnostou, A., Loukopoulos, D. & Papadakis, M. N. The Cretan type of non-deletional hereditary persistence of fetal hemoglobin [A gamma-158C->T] results from two independent gene conversion events. Hum. Genet. 102, 629-634 (1998).
20. Indrak, K. et al. Compound heterozygosity for a beta zero-thalassemia (frameshift codons 38/39; -C) and a nondeletional Swiss type of HPFH (A------C at NT-110, G gamma) in a Czechoslovakian family. Ann. Hematol. 63, 111-115 (1991).
21 Cavazzana, M., Antoniani, C. & Miccio, A. Gene Therapy for beta-Hemoglobinopathies. Mol Ther 25, 1142-1154, doi:10.1016/j.ymthe.2017.03.024 (2017).
22 Akinsheye, I. et al. Fetal hemoglobin in sickle cell anemia. Blood 118, 19-27, doi:10.1182/blood-2011-03-325258 blood-2011-03-325258 [pii] (2011).
23 Steinberg, M. H., Chui, D. H., Dover, G. J., Sebastiani, P. & Alsultan, A. Fetal hemoglobin in sickle cell anemia: a glass half full? Blood 123, 481-485, doi:10.1182/blood-2013-09-528067 (2014).
24 DeWitt, M. A. et al. Selection-free genome editing of the sickle mutation in human adult hematopoietic stem/progenitor cells. Sci Transl Med 8, 360ra134, doi:10.1126/scitranslmed.aaf9336 (2016).
25 Hoban, M. D. et al. CRISPR/Cas9-Mediated Correction of the Sickle Mutation in Human CD34+ cells. Mol Ther 24, 1561-1569, doi:10.1038/mt.2016.148 (2016).
26 Dever, D. P. et al. CRISPR/Cas9 beta-globin gene targeting in human haematopoietic stem cells. Nature 539, 384-389, doi:10.1038/nature20134 (2016).
27 Truong, L. N. et al. Microhomology-mediated End Joining and Homologous Recombination share the initial end resection step to repair DNA double-strand breaks in mammalian cells. Proceedings of the National Academy of Sciences of the United States of America 110, 7720-7725, doi:10.1073/pnas.1213431110 (2013).
28 Rahmig, S. et al. Improved Human Erythropoiesis and Platelet Formation in Humanized NSGW41 Mice. Stem Cell Reports 7, 591-601, doi:10.1016/j.stemcr.2016.08.005 (2016).
29 Fiorini, C. et al. Developmentally-faithful and effective human erythropoiesis in immunodeficient and Kit mutant mice. Am J Hematol 92, E513-E519, doi:10.1002/ajh.24805 (2017).
30 Franco, R. S. et al. The effect of fetal hemoglobin on the survival characteristics of sickle cells. Blood 108, 1073-1076, doi:10.1182/blood-2005-09-008318 (2006).
31 Centis, F. et al. The importance of erythroid expansion in determining the extent of apoptosis in erythroid precursors in patients with beta-thalassemia major. Blood 96, 3624-3629 (2000).
32 Walters, M. C. et al. Stable mixed hematopoietic chimerism after bone marrow transplantation for sickle cell anemia. Biol Blood Marrow Transplant 7, 665-673 (2001).
33 Miccio, A. et al. In vivo selection of genetically modified erythroblastic progenitors leads to long-term correction of beta-thalassemia. Proceedings of the National Academy of Sciences of the United States of America 105, 10547-10552 (2008).
34 Altrock, P. M. et al. Mathematical modeling of erythrocyte chimerism informs genetic intervention strategies for sickle cell disease. Am J Hematol 91, 931-937, doi:10.1002/ajh.24449 (2016).
35 Abraham, A. et al. Relationship between Mixed Donor-Recipient Chimerism and Disease Recurrence after Hematopoietic Cell Transplantation for Sickle Cell Disease. Biol Blood Marrow Transplant 23, 2178-2183, doi:10.1016/j.bbmt.2017.08.038 (2017).
36 Powars, D. R., Weiss, J. N., Chan, L. S. & Schroeder, W. A. Is there a threshold level of fetal hemoglobin that ameliorates morbidity in sickle cell anemia? Blood 63, 921-926 (1984).
38 Komor, A. C., Badran, A. H. & Liu, D. R. Editing the Genome Without Double-Stranded DNA Breaks. ACS Chem Biol 13, 383-388, doi:10.1021/acschembio.7b00710 (2018).
39 Lux, C. T. et al. TALEN-Mediated Gene Editing of HBG in Human Hematopoietic Stem Cells Leads to Therapeutic Fetal Hemoglobin Induction. Molecular therapy. Methods & clinical development 12, 175-183 (2019).
40 Wu, Y. et al. Highly efficient therapeutic gene editing of human hematopoietic stem cells. Nat Med, doi:10.1038/s41591-019-0401-y (2019).

1. A method for increasing fetal hemoglobin content in a eukaryotic cell comprising the step of disrupting the binding site for Leukemia/lymphoma-related factor (LRF) in the HBG1 or HBG2 promoter.

2. The method of claim 1 wherein the eukaryotic cell is selected from the group consisting of hematopoietic progenitor cells, hematopoietic stem cells (HSCs), and pluripotent cells.

3. The method of claim 1 which comprises contacting the eukaryotic cell with an effective amount of a DNA-targeting endonuclease whereby the DNA-targeting endonuclease cleaves the genomic DNA of the cell in at least one position located in or close to the binding site for Leukemia/lymphoma-related factor (LRF) in the HBG1 or HBG2 promoter.

4. The method of claim 3 wherein the DNA-targeting endonuclease leads to the genome editing of the −200 region in the HBG1 or HBG2 promoter.

5. The method of claim 3 wherein the DNA targeting endonuclease cleaves the genomic sequence
between positions −198 and −197 in the HBG1 or HBG2 promoter wherein positions −198 and −197 correspond to positions 13 and 14 in SEQ ID NO:1, or
between positions −197 and −196 in the HBG1 or HBG2 wherein positions −197 and −196 correspond to positions 14 and 15 in SEQ ID NO:1, or
between positions −196 and −195 in the HBG1 or HBG2 promoter wherein positions −196 and −195 correspond to positions 15 and 16 in SEQ ID NO:1.

6. The method of claim 3 wherein the DNA targeting endonuclease is a TALEN or a ZFN.

7. The method of claim 3 wherein the DNA targeting endonuclease is a CRISPR-associated endonuclease.

8. The method of claim 7 wherein the CRISPR-associated endonuclease is a Cas9 nuclease or is Cpf1 nuclease or any variant of these nucleases.

9. The method of claim 7 which comprises the step of contacting the eukaryotic cell with an effective amount of the CRISPR-associated endonuclease and with one or more guide RNAs.

10. The method of claim 9 wherein the one or more guide RNAs comprises:
the spacer sequence as set forth in SEQ ID NO: 2 (5′ AUUGAGAUAGUGUGGGGAAG 3′) for recruiting the CRISPR-associated endonuclease to the HBG1 and HBG2 promoters and generating double-strand breaks between positions −198 and −197 wherein positions −198 and −197 correspond to positions 13 and 14 in SEQ ID NO:1, or
the spacer sequence as set forth in SEQ ID NO: 3 (5′ CAUUGAGAUAGUGUGGGGAA 3′) for recruiting the CRISPR-associated endonuclease to the HBG1 and HBG2 promoters and generating double-strand breaks between positions −197 and −196 wherein positions −197 and −196 correspond to positions 14 and 15 in SEQ ID NO:1, or
the spacer sequence as set forth in SEQ ID NO: 4 (5′ GCAUUGAGAUAGUGUGGGGA 3′) for recruiting the CRISPR-associated endonuclease to the HBG1 and HBG2 promoters and generating double-strand breaks between positions −195 and −196 wherein positions −195 and −196 correspond to positions 15 and 16 in SEQ ID NO:1.

11. The method of claim 10 wherein the CRISPR-associated endonuclease is pre-complexed with a guide RNA to form a ribonucleoprotein (RNP) complex.

12. A method for increasing fetal hemoglobin levels in a subject in need thereof, the method comprising administering to the subject a therapeutically effective amount of a population of eukaryotic cells obtained by the method according to claim 1.

13. The method of claim 12 wherein the subject suffers from sickle cell disease or β-thalassemia.

14. A kit of parts comprising i) a CRISPR-associated endonuclease and ii) a guide RNA that comprises the sequence as set forth in SEQ ID NO:2, SEQ ID NO:3, or SEQ ID NO:4.

15. A method for the treatment of a hemoglobinopathy in a subject in need thereof, comprising, administering to the subject a therapeutically effective amount of a population of eukaryotic cells obtained by the method according to claim 1.

16. The method of claim 2 wherein the pluripotent cells are embryonic stem cells (ES) or induced pluripotent stem cells (iPS).
